(19) United States
(12) Patent Application Publication
(10) Pub. No.: US 2022/0218771 A1
(43) Pub. Date: Jul. 14, 2022

(54) METHODS AND MATERIALS FOR TREATING CARDIOVASCULAR DISEASES

(71) Applicants: Mayo Foundation for Medical Education and Research, Rochester, MN (US); Zhejiang ShouXianGu Pharmaceutical Co., Ltd., Zhejiang (CN)

(72) Inventors: Jordan D. Miller, Rochester, MN (US); Bin Zhang, Rochester, MN (US); Xinyan Liu, Beijing (CN)

(21) Appl. No.: 17/610,181

(22) PCT Filed: May 11, 2020
(86) PCT No.: PCT/US2020/032388

§ 371 Date: Nov. 10, 2021

Related U.S. Application Data
(60) Provisional application No. 62/846,452, filed on May 10, 2019.

(51) Int. Cl. A61K 36/074; A61P 9/00

(52) U.S. Cl. CPC A61K 36/074; A61P 9/00

(57) ABSTRACT

This document provides methods and materials involved in identifying and/or treating mammals having a cardiovascular disease. For example, this document provides methods and materials for administering a composition comprising a Ganoderma lucidum extract to a mammal identified as having or as being at risk of having cardiovascular disease. This document also provides methods and materials for slowing the progression of an age-related, acquired, or congenital cardiac dysfunction also are provided.

### CROSS-REFERENCE TO RELATED APPLICATIONS

This application claims priority to U.S. Patent Application No. 62/846,452, filed May 10, 2019. The disclosure of the prior application is considered part of the disclosure of this application, and is incorporated in its entirety into this application.

### BACKGROUND

### 1. Technical Field

This document provides methods and materials involved in treating mammals having a cardiovascular disease. For example, this document provides methods and materials for administering a composition containing a Ganoderma lucidum to a mammal identified as having or as being at risk of having or developing cardiovascular disease. This document also provides methods and materials for slowing the progression of age-related, acquired, or congenital cardiovascular dysfunction.

### 2. Background Information

Cardiovascular disease is the general term for heart, heart valves, and blood vessel diseases, including coronary heart disease, rheumatic and congenital heart disease, venous thromboembolism, atherosclerosis, heart valve disease, cerebrovascular disease, aorto-illiac disease and peripheral vascular disease (Steward et al., JRSM Cardiovasc. Dis., 6:1-9 (2017)). Subjects with cardiovascular disease may develop a number of complications such as myocardial infarction, stroke, angina pectoris, transient ischemic attacks, congestive heart failure, aortic aneurysm, severe valvular stenosis or regurgitation, and death. Cardiovascular disease accounts for one in every two deaths in the United States. Thus, treatment and prevention of cardiovascular disease is an area of major public health importance.

### SUMMARY

This document provides methods and materials involved in treating mammals having a cardiovascular disease. For example, this document provides methods and materials for administering a composition containing a Ganoderma lucidum (GL) extract to a mammal identified as having or as being at risk of having or developing cardiovascular disease (e.g., age-related, acquired, or congenital cardiac, vascular, or valvular dysfunction). This document also provides methods and materials for administering a composition containing a GL extract to a mammal to slow the progression of an age-related cardiac dysfunction. Having the ability to administer a composition having one or more GL extracts to treat a cardiovascular disease and/or to slow the progression of age-related, acquired, or congenital cardiac dysfunction as described herein can allow clinicians and patients to proceed with effective treatments.

In general, one aspect of this document features a method for treating a mammal having cardiovascular disease. The method comprises (or consists essentially of or consists of) (a) identifying a mammal as being in need of a treatment with a composition comprising a Ganoderma lucidum extract to treat the cardiovascular disease, and (b) administering the composition to the mammal. The mammal can be a human. The cardiovascular disease can be age-related cardiac dysfunction. The cardiovascular disease can be acquired cardiac dysfunction. The cardiovascular disease can be congenital cardiac dysfunction. The identifying step can comprise determining that the mammal comprises one or more symptoms of cardiovascular disease that are responsive to treatment with the composition. The identifying step can comprise determining that the mammal is at risk of developing one or more symptoms of cardiovascular disease that are responsive to treatment with the composition.

In another aspect, this document features a method for treating a mammal having cardiovascular disease. The method comprises (or consists essentially of or consists of) administering a composition comprising a Ganoderma lucidum extract to a mammal identified as having or as being at risk of developing a cardiovascular disease that comprises one or more symptoms that are responsive to treatment with the composition. The mammal can be a human. The cardiovascular disease can be age-related cardiovascular dysfunction. The cardiovascular disease can be acquired cardiac dysfunction. The cardiovascular disease can be congenital cardiac dysfunction.

In another aspect, this document features a method for slowing development of age-related cardiovascular dysfunction within a mammal. The method comprises (or consists essentially of or consists of) (a) identifying the mammal as being in need of treatment with a composition comprising a Ganoderma lucidum extract to slow development of the age-related cardiovascular dysfunction, and (b) administering the composition to the mammal. The mammal can be a human.

In another aspect, this document features a method for slowing development of age-related cardiovascular dysfunction. The method comprises (or consists essentially of or consists of) administering a composition comprising a Ganoderma lucidum extract to a mammal identified as being in need of a treatment to slow development of the age-related cardiovascular dysfunction. The mammal can be a human. The mammal that was identified can have one or more symptoms of age-related cardiovascular dysfunction responsive to treatment with the composition.

In another aspect, this document features a method for slowing development of acquired cardiovascular dysfunction within a mammal. The method comprises (or consists essentially of or consists of) (a) identifying the mammal as being in need of treatment with a composition comprising a Ganoderma lucidum extract to slow development of the acquired cardiovascular dysfunction, and (b) administering the composition to the mammal. The mammal can be a human.

In another aspect, this document features a method for slowing development of acquired cardiovascular dysfunction. The method comprises (or consists essentially of or consists of) administering a composition comprising a Ganoderma lucidum extract to a mammal identified as being in need of a treatment to slow development of the acquired cardiovascular dysfunction. The mammal can be a human. The mammal that was identified can have one or more symptoms of acquired cardiovascular dysfunction responsive to treatment with the composition.

In another aspect, this document features a method for slowing development of congenital cardiovascular dysfunction within a mammal. The method comprises (or consists essentially of or consists of) (a) identifying the mammal as being in need of treatment with a composition comprising a Ganoderma lucidum extract to slow development of the congenital cardiovascular dysfunction, and (b) administering the composition to the mammal. The mammal can be a human.

In another aspect, this document features a method for slowing development of congenital cardiovascular dysfunction. The method comprises (or consists essentially of or consists of) administering a composition comprising a Ganoderma lucidum extract to a mammal identified as being in need of a treatment to slow development of the congenital cardiovascular dysfunction. The mammal can be a human. The mammal that was identified can have one or more symptoms of congenital cardiovascular dysfunction responsive to treatment with the composition.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-C. Ganoderma lucidum (GL) treatment leads to reduction in severity of aortic valve stenosis. A: Experimental schematic shows that from 2 months to 11 months of age LDLR^{-/-}/apoB^{100/100} mice were given either a western diet only (“WD”) or a western diet supplemented with GL (“GL”). B: Cusp separation distance (mm) was increased with GL treatment at multiple time points (* denotes p<0.05). C: Peak velocity (mm/sec) was reduced with GL treatment at multiple time points (* denotes p<0.05).

FIGS. 2A-2C. GL treatment leads to improvement in left ventricular contractile function. A: Experimental schematic shows that from 2 months to 11 months of age LDLR^{-/-}/apoB^{100/100} mice were given either a WD or GL. B: Ejection fraction was increased with GL treatment (* denotes p<0.05). C: Global longitudinal strain was reduced with GL treatment (p<0.05) suggesting improvement in LV systolic function.

FIG. 3A-3C. GL treatment leads to improvement in left ventricular systolic/contractile function. A: Experimental schematic shows that from 2 months to 11 months of age LDLR^{-/-}/apoB^{100/100} mice were given either a WD or GL. B: Global circumferential strain decreased with GL treatment (p<0.01). C: Radial strain increased with GL treatment (p=0.08). The directionality of change with GL treatment suggests improvement in LV function in both figures.

FIG. 4A-4C. GL treatment leads to improvement in left ventricular relaxation/diastolic function and/or reduction in ventricular diastolic stiffness. A: Experimental schematic shows that from 2 months to 11 months of age LDLR^{-/-}/apoB^{100/100} mice were given either a WD or GL. B: Mitral peak velocity of early filing (E) to early diastolic mitral annular velocity (e′) or “E/e′” ratio decreased with GL treatment (* denotes p<0.001) which is consistent with improved relaxation. C: Reverse longitudinal strain rate increased with GL treatment (p<0.001) which is consistent with improved LV relaxation.

FIG. 5A-5C. GL treatment leads to improvement in left ventricular mass consistent with prevention of the maladaptive hypertrophic response to chronic left ventricular overload commonly observed in patients and animals with aortic valve stenosis. A: Experimental schematic shows that from 2 months to 11 months of age LDLR^{-/-}/apoB^{100/100} mice were given either a WD or GL. B: Left ventricular mass measured by echocardiographic measurement decreased with GL treatment (* denotes p<0.05). C: Overall cardiac mass measured by whole heart wet weight was reduced by GL (p<0.05).

FIG. 6A-D. GL treatment leads to improvement in endothelial function upon exposure to acetylcholine that is associated with reduced cardiovascular morbidity and mortality. A: Experimental schematic shows that from 2 months to 11 months of age LDLR^{-/-}/apoB^{100/100} mice were given either a WD or GL. B: Endothelium-dependent relaxation upon exposure to acetylcholine improved with GL treatment following 3 months of treatment (e.g., equivalent of early stage atherosclerosis, * denotes p<0.05). C: Endothelium-dependent relaxation upon exposure to acetylcholine improved with GL treatment following 6 months of treatment (e.g., equivalent of moderate atherosclerosis, * denotes p<0.05). D: Endothelium-dependent relaxation upon exposure to acetylcholine improved with GL treatment following 9 months of treatment (e.g., equivalent of severe atherosclerosis, * denotes p<0.05). Note that long-term treatment with GL significantly improved endothelial function in hypercholesterolemic mice and nearly completely prevented time-dependent impairments in endothelial function, an effect that is ubiquitously associated with reduced cardiovascular morbidity and mortality.

FIG. 7A-D. GL treatment leads to improvement in vasomotor function through improved responsiveness of vascular smooth muscles to nitric oxide. A: Experimental schematic shows that from 2 months to 11 months of age LDLR^{-/-}/apoB^{100/100} mice were given either a WD or GL. B: Endothelium-independent relaxation to the nitric oxide donor sodium nitroprusside was not impaired in the earliest stages of atherosclerosis (3 months of treatment). C: Endothelium-independent relaxation is impaired with 6 months of WD treatment (moderate atherosclerosis), but significantly improved with GL treatment (* denotes p<0.05). D: Endothelium-independent relaxation upon exposure to sodium nitroprusside is significantly impaired in WD mice at the 9 month time point, and is almost completely normalized by treatment with GL (* denotes p<0.05).

FIG. 8A-G. GL treatment leads to changes in vascular response to contractile agonists. A: Experimental schematic shows that from 2 months to 11 months of age LDLR^{-/-}/apoB^{100/100} mice were given either a WD or GL. B: Vascular contraction (g) after exposure to agonist Prostaglandin F_{2α} (PGF_{2α}) increased upon treatment with GL following three months of treatment compared to WD (* denotes p<0.05). C: Vascular contraction (g) after exposure to agonist Prostaglandin F_{2α} (PGF_{2α}) paradoxically decreased upon treatment with GL following 6 months of treatment compared to WD (* denotes p<0.05). D: Vascular contraction (g) after exposure to agonist Prostaglandin F_{2α} (PGF_{2α}) increased upon treatment with GL following 9 months of treatment compared to WD (* denotes p<0.05). E: Vascular contraction (g) after exposure to agonist Serotonin (5-HT) increased upon treatment with GL for 3 months compared to WD (* denotes p<0.05). F: Vascular contraction (g) after exposure to agonist Serotonin (5-HT) was unchanged upon treatment with GL for 6 months compared to WD. G: Vascular contraction (g) after exposure to agonist Serotonin (5-HT) increased upon treatment with GL for 9 months compared to WD (* denotes p<0.05). Altogether, these data suggest that contractile function of vascular smooth muscle cells is dramatically improved by long-term treatment of GL and can functionally reverse the negative impact of prolonged WD.

FIG. 9A-E. GL treatment leads to changes in intimal plaque collagen thickness (stained with picrosirius red and imaged with circularly polarized light). A: Experimental schematic shows that from 2 months to 11 months of age LDLR^{-/-}/apoB^{100/100} mice were given either a WD or GL. B: The fraction of thin collagen fibers increased in 9 month old mice treated with GL. C-D: Intermediate thickness fibers (Yellow/Orange) were largely unchanged in mice treated with GL, although there was a tendency for relatively thicker fibers to be reduced after GL treatment. E: The proportion of thick fibers was reduced in mice receiving GL for 9 months (* denotes p<0.05). Collectively, these data suggest that GL can reverse and/or attenuate some of the deleterious changes in collagen structure in advancing atherosclerosis.

FIG. 10A-C. GL treatment reduces overall plaque size but leads to substantial changes in intimal plaque calcification (stained with Alizarin Red and imaged with brightfield microscopy). A: Experimental schematic shows that from 2 months to 11 months of age LDLR^{-/-}/apoB^{100/100} mice were given either a WD or GL. B: Intimal plaque was reduced following 6 and 9 months of GL compared to age-matched littermates. C: Intimal plaque calcification was significantly reduced in mice with advanced atherosclerosis (9 month time point) receiving GL treatment (* denotes p<0.05).

FIG. 11A-B. GL treatment results in modest increases in alpha smooth muscle actin to offset pathological changes during progression of atherosclerosis (qRT-PCR). A: Experimental schematic shows that from 2 months to 11 months of age LDLR^{-/-}/apoB^{100/100} mice were given either a WD or GL. B: α-SMA is reduced with long-term WD feeding and consistent with pathological smooth muscle de-differentiation, a phenomenon which is partially offset by GL at 6 and 9 months. While subtle, this is broadly consistent with the functional contractile data presented in FIG. 8.

FIG. 12A-C. Changes in endothelial nitric oxide synthase and NADPH oxidase 2 with or without GL treatment during progression of atherosclerosis (qRT-PCR). A: Experimental schematic shows that from 2 months to 11 months of age LDLR^{-/-}/apoB^{100/100} mice were given either a WD or GL. B: Long-term treatment with GL did not restore eNOS expression at any time point. C. Long-term treatment with GL modestly reduced NOX2 expression only following 9 months of treatment in hypercholesterolemic mice.

FIG. 13A-B. GL leads to improvements in basal reactive oxygen species levels in early and late atherosclerosis (lucigenin-enhanced chemiluminescence). A: Experimental schematic shows that from 2 months to 11 months of age LDLR^{-/-}/apoB^{100/100} mice were given either a WD or GL. B: Long-term treatment with GL resulted in slight but not significant reductions in reactive oxygen species levels in aorta following 3 or 9 months of treatment with GL, suggesting that active suppression of reactive oxygen species levels are only a minor contributor to improved endothelial function following chronic GL treatment.

FIG. 14A-D. GL leads to improvements in NADPH oxidase activity in intermediate/moderate atherosclerosis (NADPH-stimulated lucigenin-enhanced chemiluminescence). A: Experimental schematic shows that from 2 months to 11 months of age LDLR^{-/-}/apoB^{100/100} mice were given either a WD or GL. B: Long-term treatment with GL for 3 months did not result in reductions in NADPH oxidase activity. C: Long-term treatment with GL resulted in modest reductions in NADPH oxidase activity in aorta following 6 months of treatment. D: Long-term treatment with GL did not result in reductions in NADPH oxidase activity following 9 months of treatment.

FIG. 15A-C. GL leads to isoform-dependent changes in matrix metalloproteinase activity in advanced atherosclerosis. A: Experimental schematic shows that from 2 months to 11 months of age LDLR^{-/-}/apoB^{100/100} mice were given either a WD or GL. B: Long-term treatment with GL reduces MMP2 expression in the most advanced stages of atherosclerotic disease (i.e., following 9 months of treatment) (* denotes p<0.05 compared to age-matched WD group). C: GL does not consistently or significantly impact expression of MMP9 during progression of atherosclerosis following 3 to 9 months of treatment compared to WD.

FIG. 16A-C. GL leads to altered fibrogenic signaling in atherosclerosis. A: Experimental schematic shows that from 2 months to 11 months of age LDLR^{-/-}/apoB^{100/100} mice were given either a WD or GL. B: GL increases TGFbeta1 expression in intermediate stages of disease (6 months of treatment), which in some contexts can reduce expression of inflammatory genes such as iNOS (* denotes p<0.05). C: COL1A1 expression is consistently reduced in early and intermediate stages of disease in GL-treated mice.

FIG. 17A-C. GL leads to altered inflammatory signaling in atherosclerosis. A: Experimental schematic shows that from 2 months to 11 months of age LDLR^{-/-} /apoB^{100/100} mice were given either a WD or GL. B: GL does not consistently or significantly alter TNFα across all time points measured. C: Inducible nitric oxide synthase (iNOS, an inflammatory gene) expression is reduced in intermediate stages of disease in GL-treated mice (* denotes p<0.05).

FIG. 18A-B. GL leads to reductions in senescent cell burden in mice with advanced atherosclerosis. A: Experimental schematic shows that from 2 months to 11 months of age LDLR^{-/-}/apoB^{100/100} mice were given either a WD or GL. B: GL reduces p16^{ink4a} expression (a key marker of cellular senescence) in aorta from hypercholesterolemic mice.

FIG. 19A-D. GL does not consistently influence mRNA levels of key genes related to ectopic osteogenesis in atherosclerosis. A: Experimental schematic shows that from 2 months to 11 months of age LDLR^{-/-}/apoB^{100/100} mice were given either a WD or GL. B: Bone morphogenetic protein 2 (a major driver of ectopic calcification in cardiovascular tissues) is not reduced by long-term treatment with GL. C: Runx2 (a master regulator of osteogenesis that is commonly induced with chronic BMP2 elevations) is not reduced by long-term treatment with GL. D: Osterix (a transcription factor often induced by BMP2 signaling) is slightly reduced in early atherosclerosis, but not significantly reduced in later stages of atherosclerotic disease.

FIG. 20A-C. GL does not consistently influence mRNA levels of key genes related to ectopic calcification in atherosclerosis. A: Experimental schematic shows that from 2 months to 11 months of age LDLR^{-/-}/apoB^{100/100} mice were given either a WD or GL. B: GL does not alter expression of SPP1 in early/intermediate disease stages, but mildly reduces SPP1 in late atherosclerosis (9 month time point). C: GL does not alter expression of ALPL across the spectrum of atherosclerotic disease.

FIG. 21A-C. GL treatment leads to improvement in left ventricular mass after normalized by bodyweight consistent with prevention of the maladaptive hypertrophic response to chronic left ventricular overload commonly observed in patients and animals with aortic valve stenosis. A: Experimental schematic shows that from 2 months to 11 months of age LDLR^{-/-}/apoB^{100/100} mice were given either a WD or GL. B: Left ventricular mass measured by echocardiographic measurement decreased with GL treatment even after normalizing by bodyweight (* denotes p<0.05). C: Overall cardiac mass measured by whole heart wet weight was not altered by GL after normalization for changes in body size.

FIG. 22A-B. GL treatment leads to increases in intimal plaque collagen levels in atherosclerotic plaques. A: Experimental schematic shows that from 2 months to 11 months of age LDLR^{-/-}/apoB^{100/100} mice were given either a WD or GL. B: Changes in total collagen burden in the intimal portion of aortic plaques following long-term treatment of hypercholesterolemic mice with Ganoderma lucidum (GL). Note that long-term treatment with GL dramatically increased total amount of collagen in the atherosclerotic plaque following 9 months of treatment in hypercholesterolemic mice, which would generally be consistent with stabilization of a lipid-rich atherosclerotic plaque.

FIG. 23 A-D. GL does not consistently influence mRNA levels of key genes related to left ventricle fibrosis. A: Experimental schematic shows that from 2 months to 11 months of age LDLR^{-/-}/apoB^{100/100} mice were given either a WD or GL. B-C: Histogram shows expression patterns for matrix metalloproteinase-2 (MMP2) or matrix metalloproteinase-9 (MMP9) throughout disease progression. D: GL does consistently alter expression of Periostin (POSTN) in early/late disease stages, but reduces POSTN at intermediate time of disease (6 month time point).

FIG. 24 A-E. Changes of thickness of collagen fibers implicated in left ventricle from long-term treatment of hypercholesterolemic mice with Ganoderma lucidum. A: Experimental schematic shows that from 2 months to 11 months of age LDLR^{-/-}/apoB^{100/100} mice were given either a WD or GL. B-E: Histograms show changes for each collagen thickness throughout disease progression. GL does not alter thickness at any stage, suggesting GL did not change the composition of different thicknesses in the left ventricle throughout disease progression.

FIG. 25A-E. Changes in gene expression levels of pro-fibrotic markers in left ventricle from long-term treatment of hypercholesterolemic mice with Ganoderma lucidum. A: Experimental schematic shows that from 2 months to 11 months of age LDLR^{-/-}/apoB^{100/100} mice were given either a WD or GL. B-C: Note that there are different patterns of expression for each gene throughout disease progression TGFβ1 (panel B) and TGFβ2 (panel C). D-E: GL treatment does not consistently alter expression of COL1A1 and COL3A1 in early/late disease stages. GL does significantly reduce COL3A1 expression at the intermediate-stages of disease (6 month time point).

FIG. 26 A-B. Changes in gene expression of a senescence marker in left ventricle from long-term treatment of hypercholesterolemic mice with Ganoderma lucidum. A: Experimental schematic shows that from 2 months to 11 months of age LDLR^{-/-}/apoB^{100/100} mice were given either a WD or GL. B: GL treatment leads to significant reductions in senescent cell marker, Cyclin-Dependent Kinase Inhibitor 2A (CDKN2A or p16ink4A), in left ventricle at early/intermediate stages of disease. Note that GL does not alter expression of p16ink4A late disease stages (9 month time point).

### DETAILED DESCRIPTION

This document provides methods and materials for treating mammals having a cardiovascular disease, methods and materials for treating mammals at risk for developing a cardiovascular disease, and methods and materials for slowing the progression of age-related, acquired, or congenital cardiovascular dysfunction. For example, this document provides methods and materials for administering a composition containing one or more GL extracts to a mammal identified as having a cardiovascular disease to treat that cardiovascular disease. As used herein, the term “cardiovascular disease” refers to a class of diseases that involves the heart, heart valves, and/or blood vessels and their subcomponents (e.g., vascular/venous valves) including “cardiac dysfunction” that involves aging-related, acquired, or congenital dysfunction of the heart, heart valves, blood vessels, and/or other structures considered to be classified as the cardiovascular system. In some cases, a composition containing one or more GL extracts can be administered to a mammal identified as having or at risk of developing age-related, acquired, or congenital cardiovascular dysfunction to slow the progression of that age-related, acquired, or congenital dysfunction.

Any appropriate mammal can be identified as having or as being at risk of developing a cardiovascular disease. For example, humans and other primates such as monkeys can be identified as having or as being at risk of developing a cardiovascular disease. In some cases, dogs, cats, horses, cows, pigs, sheep, mice, or rats can be identified as having a cardiovascular disease as described herein.

Any appropriate cardiovascular disease can be treated with a composition comprising one or more GL extracts as described herein. For example, cardiovascular diseases including, without limitation, cardiomyopathy, hypertensive heart disease (e.g., related to high blood pressure), heart failure, valvular heart disease, congential heart disease, rheumatic heart disease, pulmonary heart disease, cardiac dysrhythmias, endocarditis, myocarditis, eosinophilic myocarditis, aortic aneurysm, renal artery stenosis, coronary artery disease, peripheral arterial disease, and cerebrovascular disease can be treated as described herein. In some cases, a mammal having age-associated cardiac dysfunction in cardiovascular tissue can be treated with a composition comprising one or more GL extracts as described herein.

As described herein, a mammal (e.g., a human) can be identified as having or as being at risk of having a cardiovascular disease or age-associated cardiac dysfunction by determining a cardiovascular risk score. In some cases, the risk score is determined by a history of previous cardiovascular events (e.g., stroke or heart attack). In some cases, the risk score is determined by existing cardiovascular disease. Examples of risk scores include, without limitation, ASSIGN, Framingham, QRISK, Agatson calcification score, and ASCVD risk scores. Additional examples of scores and risk factors that can be used to identify a mammal to treat as described herein include, without limitation, coronary artery calcification score, valvular calcification score, echocardiographic scoring and disease stratification, high sensitivity C-reactive protein (hs-CRP), ankle-brachial pressure index, lipoprotein(a), apolipoproteins A-I and B, fibrinogen, lipoprotein subclasses and particle concentration, homocysteine, N-terminal pro B-type natriuretic peptide (NT-proBNP), white blood cell count and markers of kidney function.

As described herein, a mammal (e.g., a human) can be identified as having cardiovascular disease by determining the function of the heart muscle. Examples of measuring the function of the heart muscle include, without limitation, measuring the electrical activity of the heart (e.g., electrocardiogram), myocardial perfusion imaging (e.g., single-photon emission computed tomography (SPECT)), unstressed cardiac imaging (e.g., resting echocardiography, gated computed tomography (CT) imaging, or magnetic resonance imaging (MRI)), and cardiac stress testing (e.g., stress echocardiography or nuclear stress test).

In some cases, a mammal can be identified as having age-associated cardiac dysfunction by determining left ventricular (LV) diastolic function, LV systolic reserve capacity, arterial stiffness, heart valve function, blood flow and/or blood vessel narrowing in various tissues, and/or endothelial cell function.

Once identified as having a cardiovascular disease, as being at risk of developing a cardiovascular disease, and/or as being in need of a treatment described herein, the mammal can be treated as described herein. For example, once a mammal is identified as being in need of a slowing of the progression of an age-related, acquired, or congenital cardiac, heart valve, or vascular dysfunction, the mammal can be administered a composition containing one or more GL extracts.

Any appropriate GL extract can be used as described herein. For example, a composition can be formulated to include a GL extract having the ingredients described in Table 1. As used herein, a “Ganoderma lucidum,” “Ganoderma lucidum extract”, “Lingzhi”, “Lingzhi Extract”, “Reishi”, “Reishi Extract”, or “GL extract” refers to a preparation of a broad composition of biologically active molecules contained within or extracted from Ganoderma lucidum source material, including any extract structure, substructure, component, or derivative/isolated subcomponent. Any appropriate Ganoderma lucidum source material can be used to produce a GL extract. For example, the entire mushroom, root, stem, cap, and/or spores can be obtained from Ganoderma lucidum and used as a therapeutic alone or used for source material to produce a GL extract.

TABLE 1

| | | Example of specific |
|---|---|---|
| Ingredients | Range | amount |
| Crude Polysaccharide | ≥8.0% | 15.9% |
| Total Triterpenes | ≥4.0% | 5.8% |
| Other | ≤88% | |

Any appropriate method can be used to produce a GL extract that can be used to make a composition provided herein. For example, a mode of delivery of any GL extract can include, but is not limited to, the direct consumption of the mushroom and/or its components in an unprocessed or processed form, ultrasonic fracturing of the spores/GL mushroom structures, CO2 and/or impact fracturing of spores/GL mushroom structures, pulverizing of the spores/GL mushroom structures to a consumable powder form, chemical degradation and extraction of spores/GL mushroom structure (including but not limited to ethanol, water, and other extract media), and/or any combination of these extraction methods can be used to make a GL structure, substructure, component, derivative/isolated subcomponent, or extract. In some cases, a GL extract can be obtained commercially. Examples include, but are not limited to, GL compositions that can be obtained from Zhejiang Shouxiangu Pharmaceutical (Ganoderma Broken Lingzhi Spore Extract, G20160355); Anhui Limin Biological Technology Co., LTD (Danhua Ganoderma lucidum Spore Powder, Approval/Cat. No. G20141225; Danhua Ganoderma lucidum oral liquid Approval/Cat. No. G20040863; Ganoderma lucidum spore oil soft capsule, Approval/Cat. No. G20120525), or Ganoherb Technology Corp. (Ganoderma lucidum Spore Oil Softgel; Ganoderma lucidum Cell-wall Broken Spore Powder, Approval/Cat. No. G20100068), Mikei (NPN/Cat. No. 80035167), Zhejiang Conba Pharmaceutical Co., Ltd. (Approval/Cat. No. G20140842) or Zhejiang Shouxiangu Pharmaceutical (Ganoderma Spore oil soft capsule, G20200107).

Once obtained, the GL extract can be used as-is, can be used to formulate a composition that includes the GL extract, or can be added to food products, such as, without limitation, meal replacers, snacks, and beverages. In some cases, a GL extract can be used in food supplements that are formulated as multivitamins, tablets, or capsules. When formulating a composition containing one or more GL extracts for use as described herein, the composition can contain any appropriate amount of the GL extract. For example, a composition can be formulated to include from <1% percent (e.g., wt/volume in a dense beverage or meal/snack) to about 99.9 percent (e.g., capsular form) of a GL extract. In some cases, a composition containing a GL extract can include the GL extract as the sole active ingredient for treating a cardiovascular disease and/or for slowing the progression of an age-related, acquired, or congenital cardiac dysfunction. In some cases, a composition can be formulated to contain one or more GL extracts and one or more other ingredients. For example, a composition containing a GL extract can include one or more other ingredients as described in Tables 2-3.

TABLE 2
Swanson 7 Mushroom Complex (Herbal Supplement)

| Ingredients | Percentage |
|---|---|
| GL Extract | 14% |
| Agaricus Blazei Extract | 14% |
| Bionectria Ochroleuca | 14% |
| Grifola Frondosa | 14% |
| Hericium erinaceus | 14% |
| Lentinula edodes | 14% |
| Coriouls Versicolor | 14% |

TABLE 3
Solgar Reishi Shitake Maitake Mushroom Extract

| Ingredients | Amount per capsule |
|---|---|
| Ganoderma Lucidum extract | 30 mg |
| Lentinula Edodes Extract | 1.5 mg |
| Grifola Frondosa Extract | 100 mg |
| Standardized Ganoderma Lucidum | 30 mg |
| Extract (4% triterpenes & 12.5% | |
| polysaccharides) | |
| Standardized Lentinula Edodes (from 1.5 | 30 mg |
| mg of 20:1 shiitake mushroom extract) | |

A composition containing a GL extract can be administered to a mammal once or multiple times over a period of time ranging from days to months or years. In some cases, a composition containing a GL extract can be formulated into a pharmaceutically acceptable composition for administration to a mammal. For example, a therapeutically effective amount of a composition containing a GL extract can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing a GL extract described herein (e.g., a pharmaceutical composition containing a GL extract active in resolving one or more symptoms of a cardiovascular disease) can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including a GL extract can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the cardiovascular disease, the route of administration, the age and general health condition of the subject, excipient usage, the possibility of co-usage with other therapeutic treatments, and the judgment of the treating physician. For example, suitable dosages of a composition containing a GL extract can be in the range of about 10 micrograms to about 1000 micrograms, about 1 milligrams to about 1000 milligrams or about 1 grams to 10 grams of the composition/day depending on the product purity, overall product composition (with or without fractured spore shell), and condition being treated.

An effective amount of a composition containing a GL extract described herein can be any amount that reduces the symptoms of cardiovascular disease within a mammal (e.g., a human) and/or that slows the progression of an age-related, acquired, or congenital cardiac dysfunction without producing severe toxicity to the mammal. For example, an effective amount of a composition containing a GL extract can be from about 25 mg to 50 mg daily. If a particular mammal fails to respond to a particular amount, then the amount of the composition administered can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cardiovascular disease) may require an increase or decrease in the actual effective amount administered.

The frequency of administration of a composition containing a GL extract described herein can be any amount that reduces the symptoms of cardiovascular disease within a mammal (e.g., a human) and/or that slows the progression of an age-related, acquired, or congenital cardiac dysfunction without producing significant toxicity to the mammal. For example, the frequency of administration of a composition containing a GL extract can be from about once a day to about once a month. The frequency of administration of the composition containing a GL extract described herein can remain constant or can be variable during the duration of treatment. A course of treatment with a composition containing a GL extract described herein can include rest periods. For example, a composition containing a GL extract can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cardiovascular disease) may require an increase or decrease in administration frequency.

In some cases, a composition containing a GL extract may be used in combination with other prophylactic or therapeutic treatments for cardiovascular disease. For example, without limitation, a composition containing a GL extract may administered along with ACE inhibitors, aldosterone inhibitors (e.g., eplerenone or spironolactone), angiotensin II receptor blockers, beta-blockers, calcium channel blockers, cholesterol lowering drugs, digoxin, diuretics, inotropic therapy, magnesium or potassium, proprotein convertase subtilisin kexin type 9 (Pcks9) inhibitors, vasodilators and/or warfarin.

An effective duration for administering a composition containing a GL extract can be any duration that reduces the symptoms of cardiovascular disease within a mammal (e.g., a human) and/or that slows the progression of an age-related, acquired, or congenital cardiac dysfunction without producing significant toxicity to the mammal. In some cases, the effective duration can vary from several days to several months to several years. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated (e.g., cardiovascular disease) can be monitored. Any appropriate method can be used to determine whether or not a mammal having cardiovascular disease is being treated. For example, clinical scanning techniques can be used to determine the presence or absence of the symptoms of cardiovascular disease within a mammal (e.g., a human) being treated.

The invention will be further described in the following examples, which do not limit the scope of the invention described in the claims.

### EXAMPLES

### Example 1—Long-Term Treatment (9 Month Treatment Duration) of LDLR^{-/-}/apoB^{100/100} Mice with Ganoderma lucidum

### Mouse Lines and Animal Husbandry

Ldlr^{-/-}/apoB^{100/100} mice were intercrossed to maintain homozygosity of mutations on both genes and generate littermate-matched, Ldlr^{-/-}/apoB^{100/100} offspring that were randomized to either control or experimental treatment groups. The LDLR^{-/-}/apoB^{100/100} mouse model has previously demonstrated to predict patient responses in Phase II clinical trials (NCT02481258: A PHASE II RANDOMIZED, PLACEBO-CONTROLLED, DOUBLE-BLINDED STUDY EVALUATING THE EFFECTS OF ATACIGUAT (HMR1766) ON AORTIC VALVE CALCIFICATION IN PATIENTS WITH MODERATE CALCIFIC AORTIC VALVE STENOSIS). Mice were placed on either a Western diet+Ganoderma lucidum (GL) or a western diet only (“WD”) and divided into the groups illustrated in the figures: “WD” or “GL”). Each group remained on the Western diet+/−GL for 9 months.

### Results

The cardiovascular system delivers oxygenated blood to all tissues in the body, and is thus involved for health of every tissue and longevity of the organism as a whole. Therefore, studying the impact of aging on the heart and the arterial system is helpful for treating age-associated cardiac dysfunction. During aging, pathological alterations in cardiovascular tissue include altered left ventricular (LV) diastolic function, and diminished LV systolic reverse capacity, increased arterial stiffness and impaired endothelial function. In this study, mice treated with GL were assessed for: aortic valve stenosis, LV diastolic contractile function, LV systolic contractile function, LV diastolic stiffness, endothelial function and smooth muscle function.

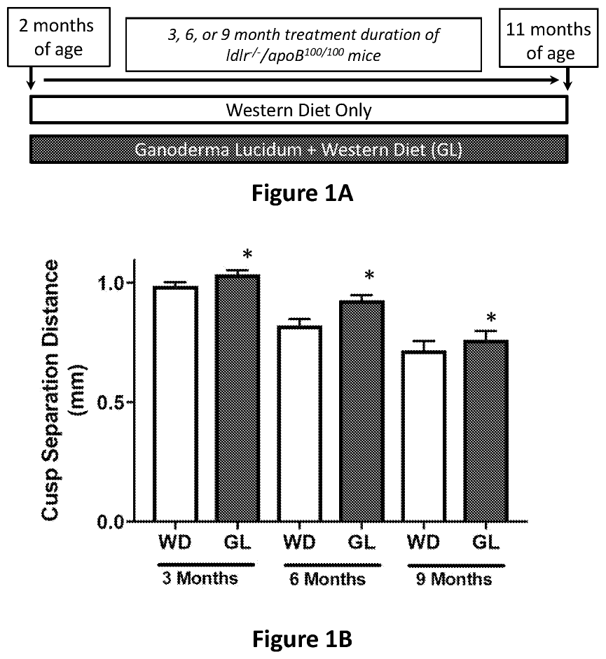

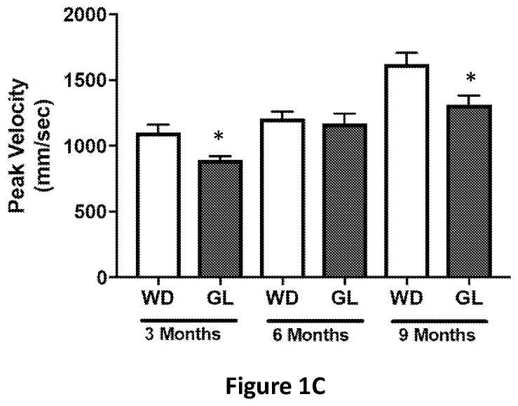

Cardiovascular stiffness accompanies age-associated cardiac dysfunction. Therefore, following 9-month treatment with GL aortic valve function was assessed using peak velocity and systolic cusp separation measurements. Those in the GL group exhibited a significant reduced peak velocity (p<0.01) (FIG. 1C) and exhibited a greater systolic cusp separation distance when compared to the WD only controls (p=0.06, FIG. 1B). Note that increases in cusp separation distance and reductions in peak trans-vascular velocity are consistent with reductions in the severity of aortic valve stenosis and a sign of improved function.

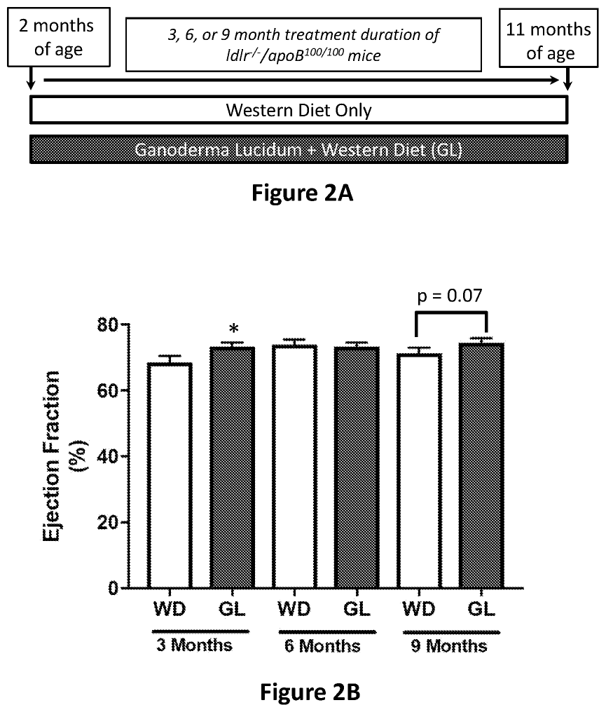

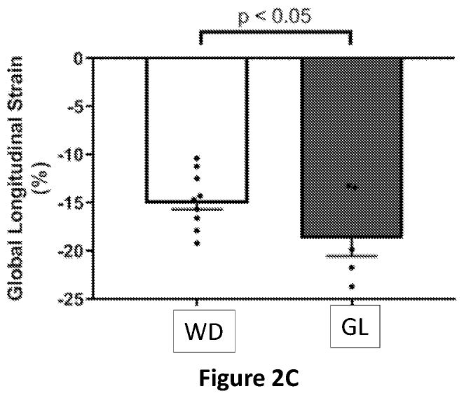

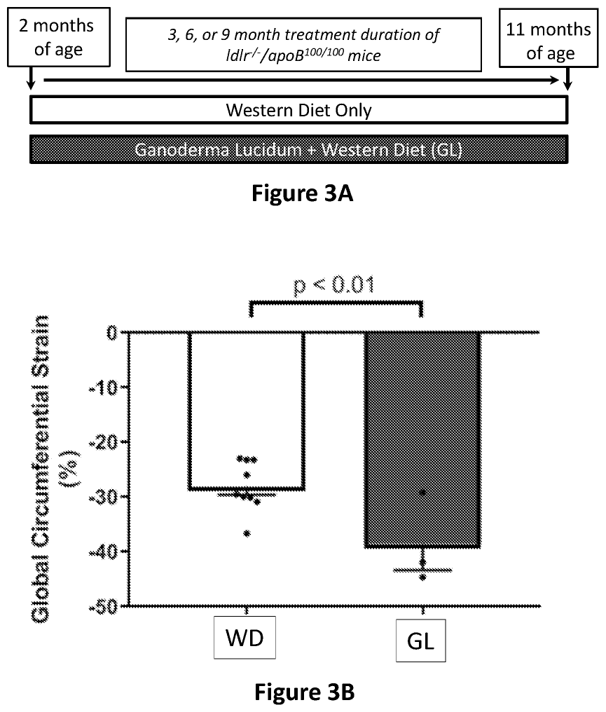

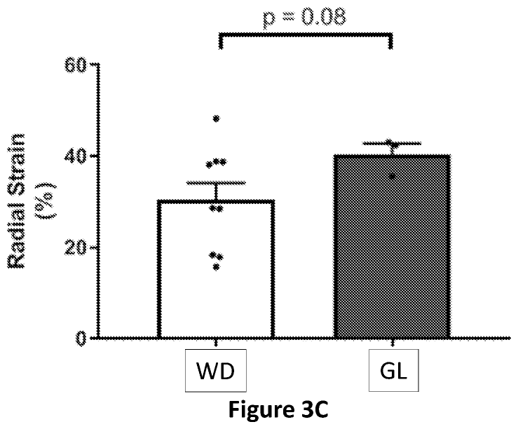

Next, assessment of left ventricular systolic and contractile function showed improvement across a range of tests. While differences in ejection fractions were not significantly different between the two groups (p<0.08), ejection fraction did increase in the GL group (FIG. 2B). In a measure of global longitudinal strain, the GL group exhibited a significant decrease (e.g., more negative) than WD controls (p<0.05, FIG. 2C). The GL group also exhibited a significant decrease (e.g., more negative) in global circumferential strain (p<0.01, FIG. 3B). Finally, increases in radial strain for the GL (p<0.08, FIG. 3C) represented the fourth piece of evidence. Collectively, all four lines of evidence suggested improved left ventricular systolic and contractile function after treatment with GL.

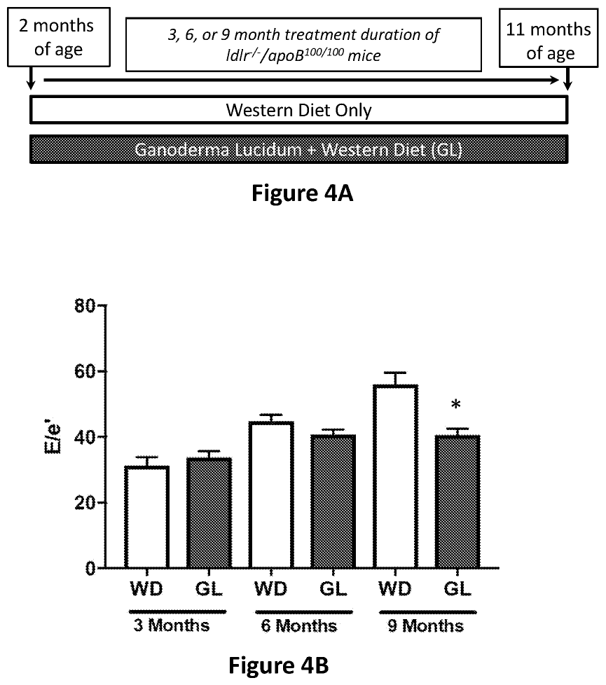

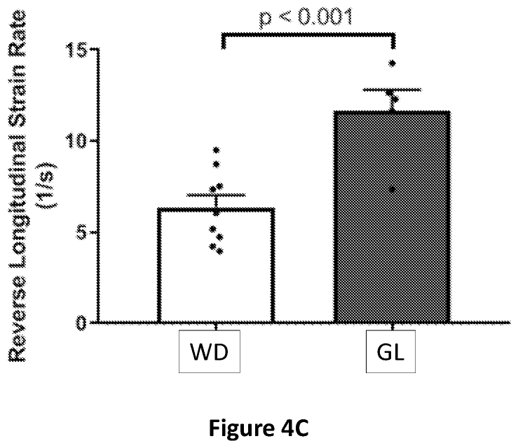

Further testing of overall LV function revealed that ventricular diastolic function improved following treatment with GL. The E/e′ ratio (Mitral peak velocity of early filing (E) to early diastolic mitral annular velocity (e′)) decreased in GL treated mice (p<0.001, FIG. 4B). In parallel, reverse longitudinal strain rate increased with GL treatment (p<0.001, FIG. 4C). Both of these findings are consistent with improvements in left ventricular relaxation/diastolic function and/or reductions in ventricular diastolic stiffness.

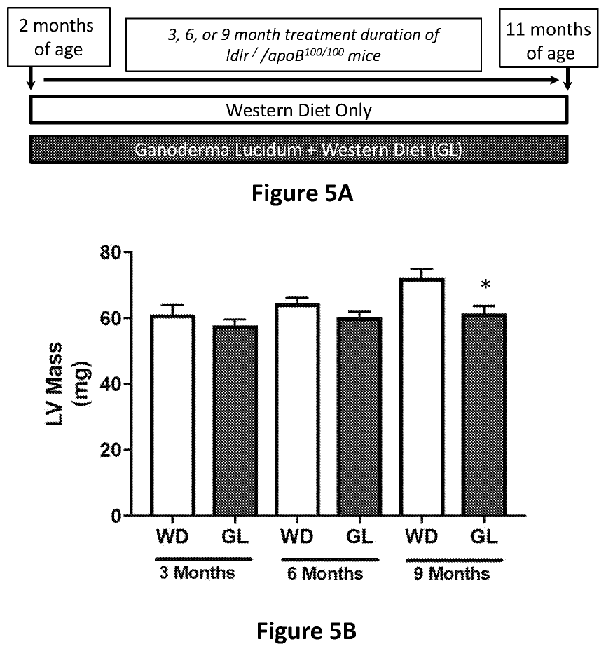

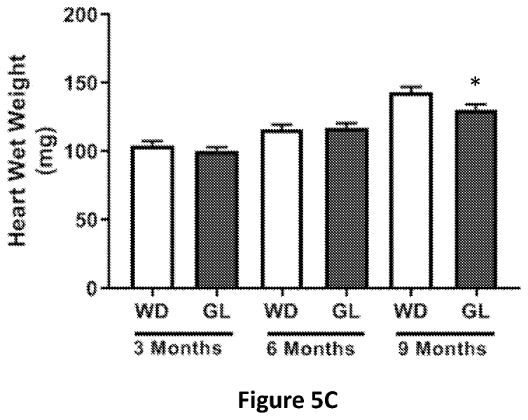

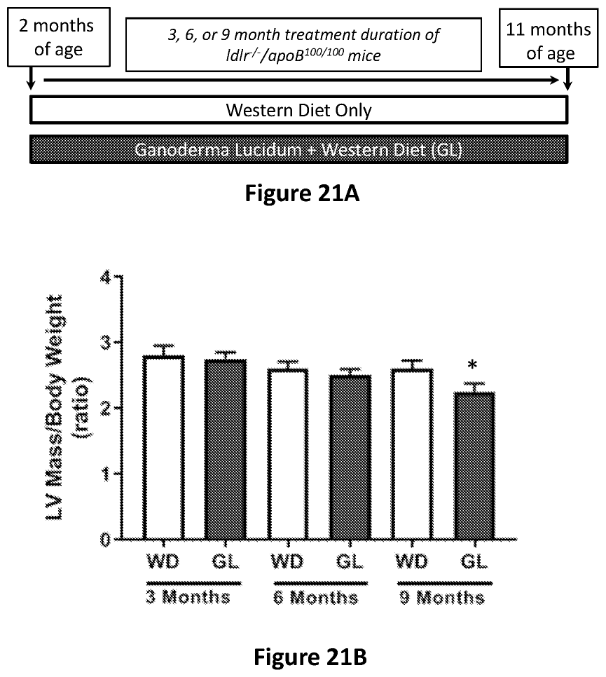

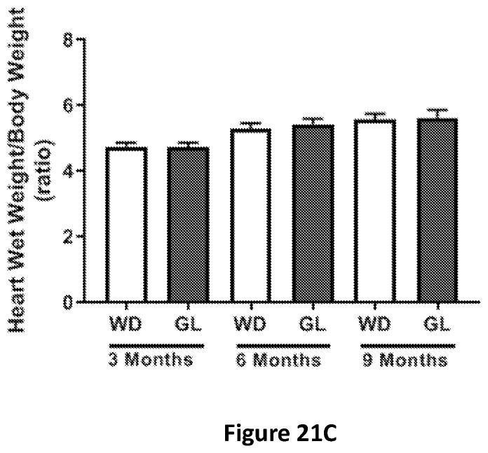

Another marker for ventricular adaptation/maladaptation is ventricular mass. In particular, ventricular thickening is associated with maladaptive hypertrophic response to chronic left ventricular overload commonly observed in patients and animals with aortic valve stenosis. Echocardiographic measurements of left ventricular mass revealed reductions in the GL treated mice (p<0.01, FIG. 5B, which persisted after normalization by bodyweight, FIG. 21B) as well as reductions in overall heart wet weight (FIG. 5C, normalized by bodyweight in FIG. 21C), suggesting that GL may aid in preventing the left ventricular maladaptive hypertrophic response.

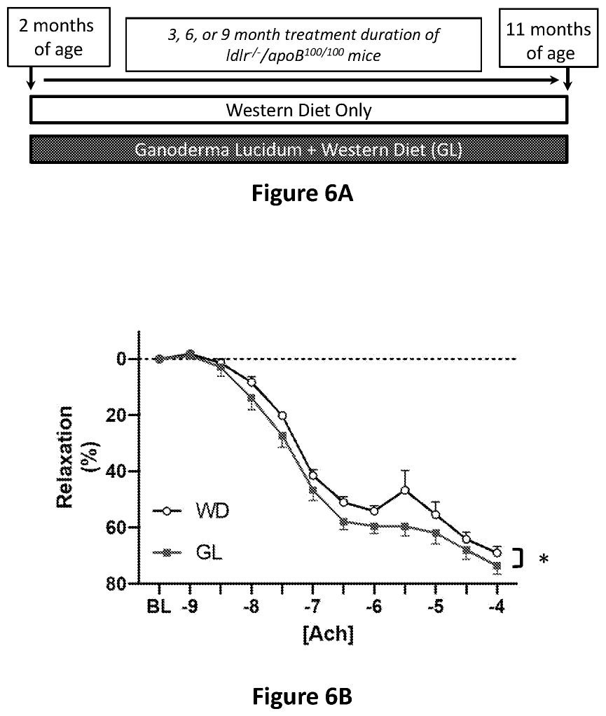

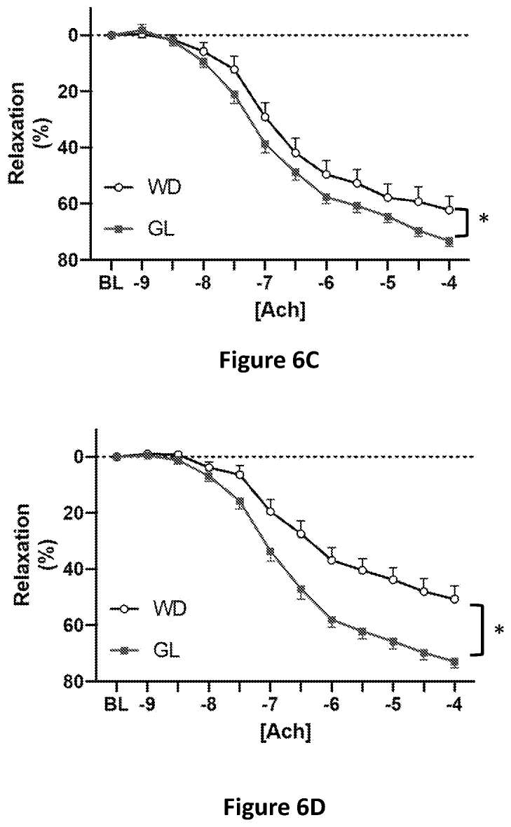

During aging, the vasculature undergoes structural and functional alterations. For example, luminal enlargement results in wall thickening that leads to a decline in endothelial cell function. The decreased function manifests as a decrease in the ability to relax in response to various physiological stimuli, which can result in increased vascular stiffness, increased thrombotic risk, and accelerated atherosclerosis and its complications. Therefore, endothelial function was assessed following treatment with GL. Measuring endothelial relaxation following acetylcholine treatment revealed that GL treatment significantly improved endothelial relaxation (p<0.05, FIG. 6B-D; 6B—3 month, 6C—6 months, 6D—9 months). The improved endothelial function in hypercholesterolemic mice was associated with reduced cardiovascular morbidity and mortality.

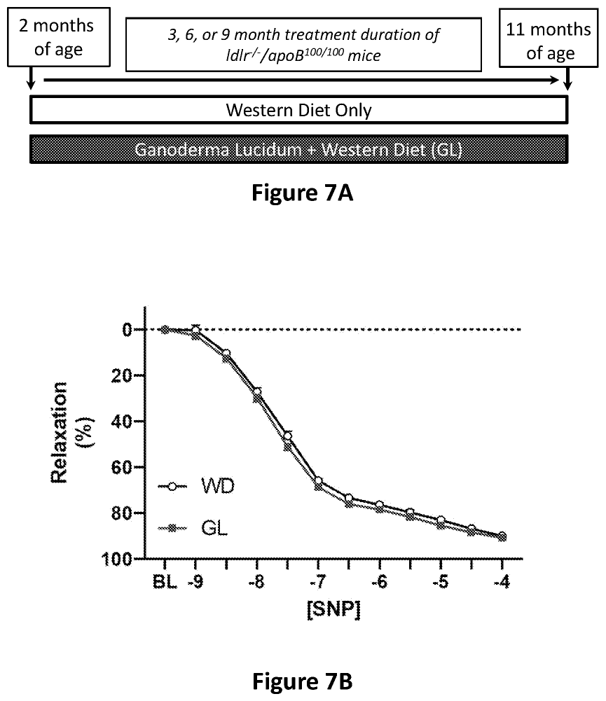

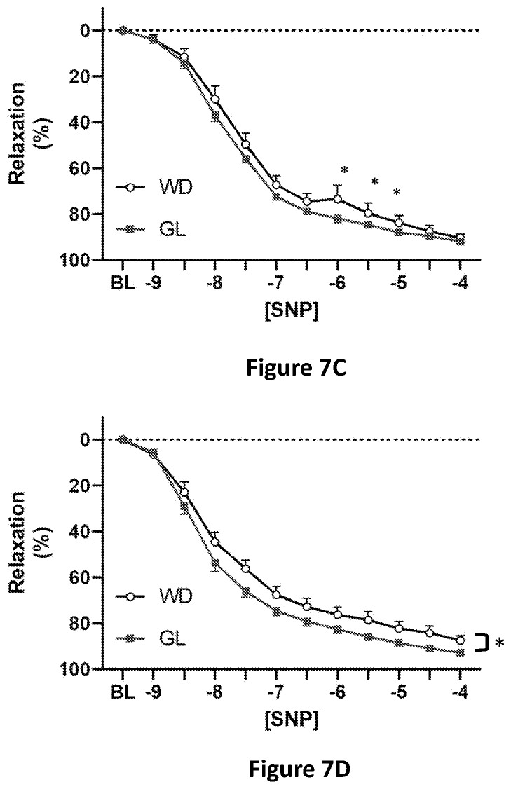

During aging, vascular smooth muscle cells can also become less sensitive to protective factors released by the endothelium (e.g., nitric oxide), which can ultimately promote increases in vascular tone, increased vascular stiffness, and accelerated vascular calcification. As a result, endothelial-independent relaxation mechanisms play an increasing role during aging. Assessment of endothelial-independent relaxation was helpful in gaining a full understanding of GL treatment. Endothelial-independent relaxation was assessed by measuring vascular smooth muscle responsiveness using a nitric oxide donor (sodium nitroprusside) and revealed that treatment with GL improved relaxation (p<0.05, FIG. 7B-D; 7B—3 months, 7C—6 months, 7D—9 months). An increase in responding vascular smooth cells illustrated increased function that complemented improvements in endothelium-dependent relaxation (FIG. 6).

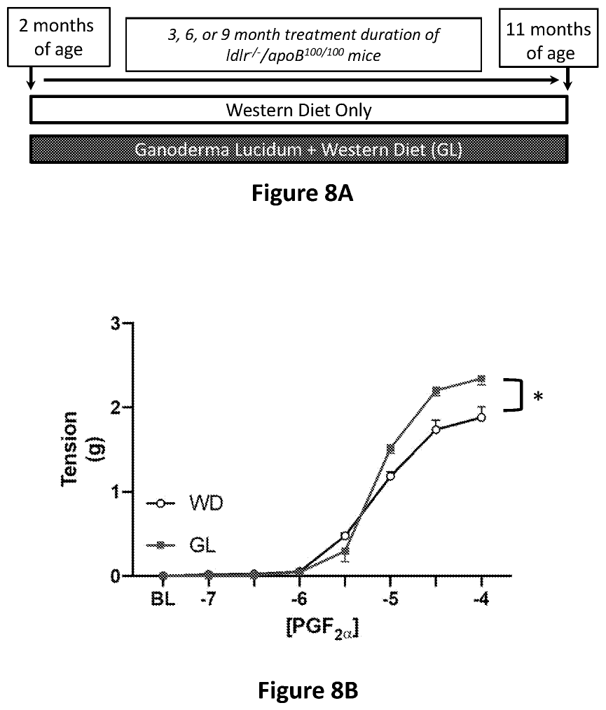

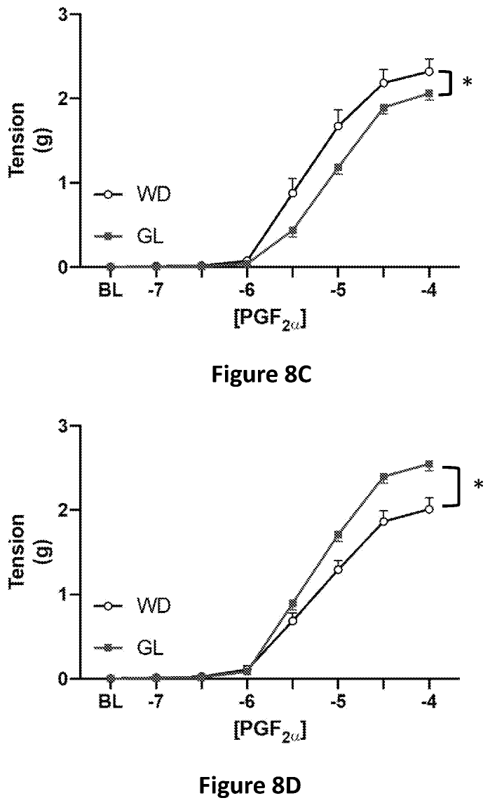

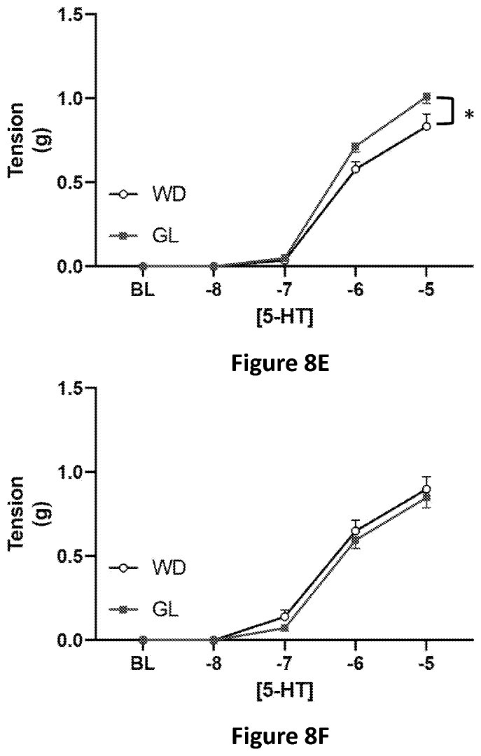

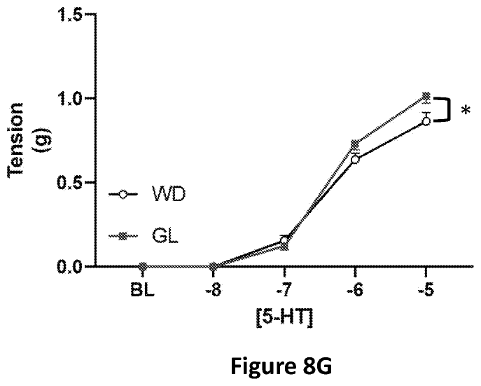

Additional importance of endothelial-independent mechanisms can be highlighted by early stages of vascular calcification and stiffening being associated with losses in vascular smooth muscle contractile protein expression and losses in force production. Measuring vascular smooth muscle contraction following treatment with contractile agonists Prostaglandin F_{2α} (FIG. 8B-D; 8B—3 months, 8C—6 months, 8D—9 months) and Serotonin (FIG. 8E-G; 8E—3 months, 8F—3 months, 8G—9 months) revealed significant increases in contraction following treatment with GL at multiple time points (p<0.05 for both). Improved contraction was concomitant with increased responsiveness of vascular smooth muscle thereby showing the impact of GL on endothelial-dependent and independent mechanisms.

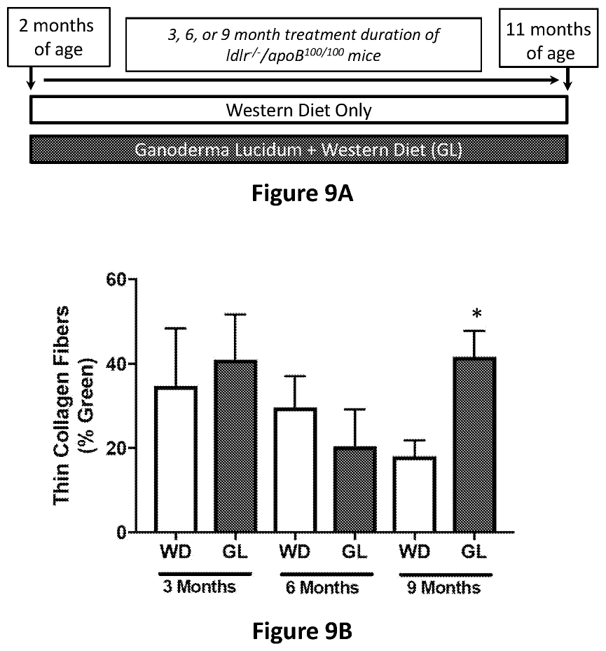

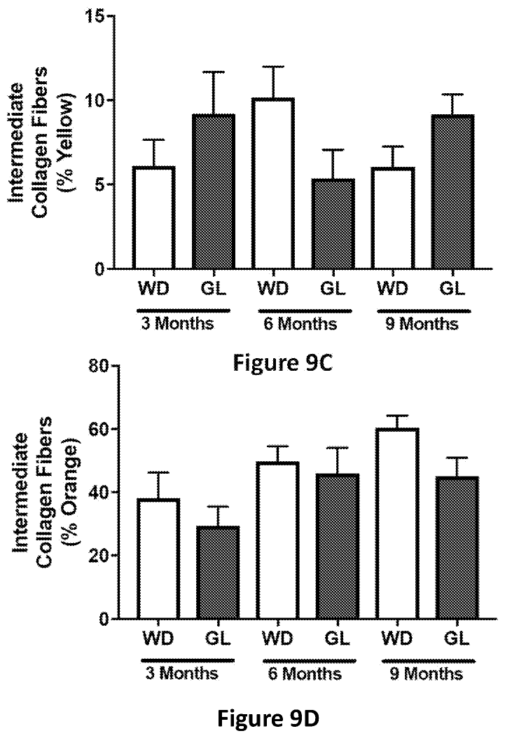

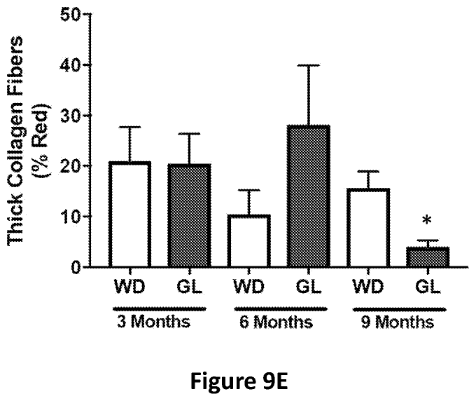

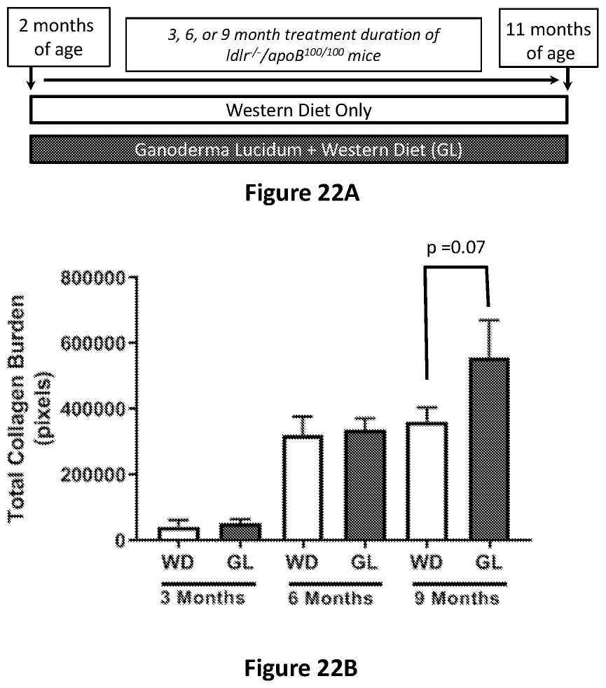

Intimal plaque fibrosis can be a major contributor to both plaque stiffness (which can augment vascular calcification) and can also be significant determinant of risk for plaque rupture and cardiovascular events. Measuring collagen fiber thickness using picrosirius red staining and circularly polarized light imaging (which allows for assessment of relative collagen fiber thicknesses) in FIG. 9B revealed that long-term treatment with GL increased the proportion of thin collagen fibers within the intimal plaque in severe atherosclerosis such so that it is more similar to an immature plaque (e.g., similar to a smaller 3 month WD lesion). This was associated with reductions in the proportion of thick fibers in GL-treated mice/lesions at the 9 month time point. As shown in FIG. 22B, however, GL may increase overall plaque fibrosis, which would stabilize lipid-rich plaques. Collectively, these structural improvements occurred concomitant with in improvements in other measurements of cardiovascular stiffness (e.g., left ventricular diastolic stiffness in FIG. 4) suggesting a broader impact of GL on cardiovascular stiffness and plaque stability in multiple tissues.

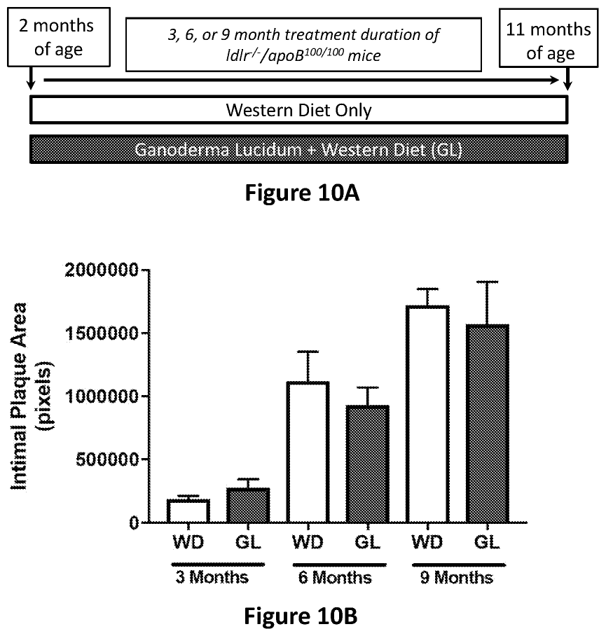

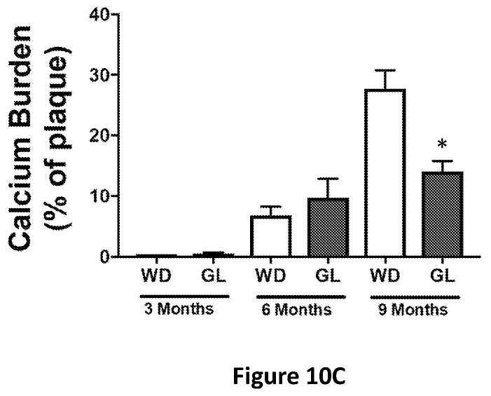

While intimal plaque size is a significant determinant of cardiovascular risk, intimal plaque composition is a major determinant of both cardiovascular risk, cardiovascular stiffness, and response to lipid lowering treatment (e.g., propensity for lesion regression). In particular, cardiovascular calcification not only imparts increased risk of morbidity and mortality but also makes plaque regression in response to lipid lowering/risk factor mitigation much less likely. Histopathological assessments of plaque size and calcific burden using Alizarin red staining (FIG. 10) revealed that long-term treatment with GL only modestly attenuates lesion size in hypercholesterolemic mice (FIG. 10B) but dramatically reduces calcium burden (FIG. 10C) in aortic plaques compared to age- and littermate-matched WD mice. Thus, these data suggest that GL is a viable strategy to reduce cardiovascular calcification and subsequent associated increases in cardiovascular risk, increases in cardiovascular stiffness, and improve lesion regression in response to aggressive lipid lowering.

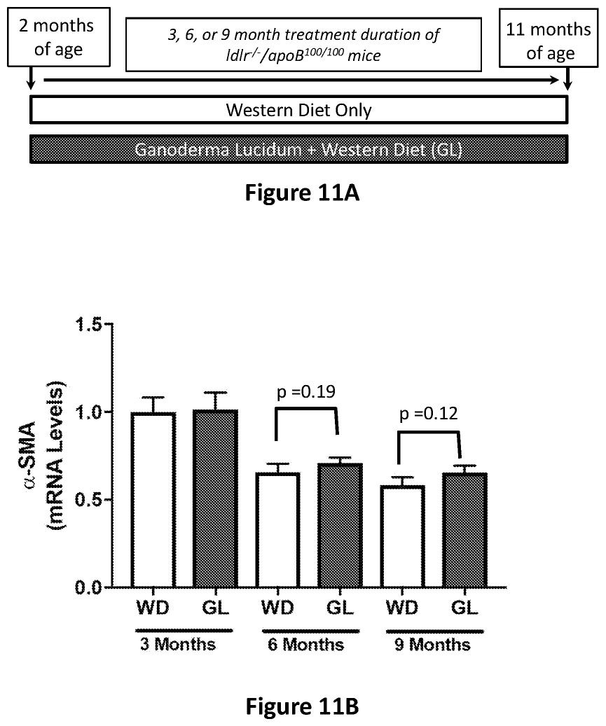

Reductions in contractile protein expression in vascular smooth muscle cells (often referred to as VSMC de-differentiation) promote vascular fibrosis and calcification. Analysis of alpha smooth muscle actin expression in aortic segments from GL-treated mice showed that alpha-SMA tended to increase with GL treatment in advanced stages of atherosclerosis (FIG. 11). This suggests GL treatment may be a viable strategy to attenuate vascular smooth muscle de-differentiation and subsequent cardiovascular morbidity/mortality in a variety of disease conditions.

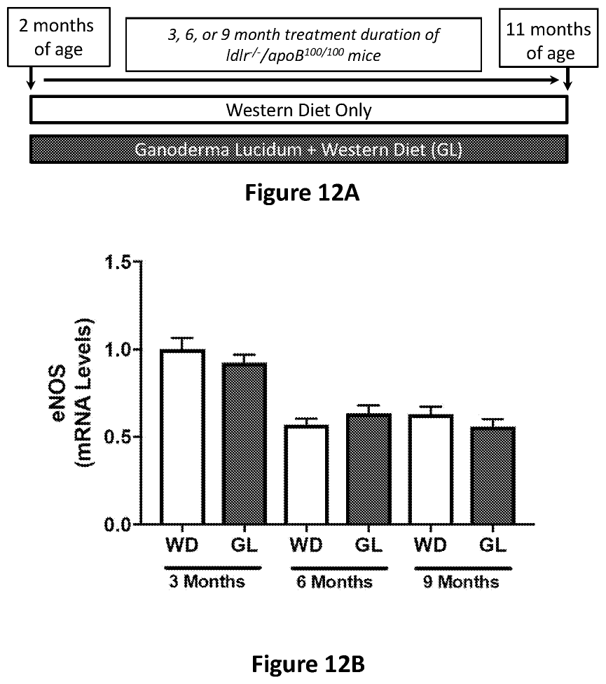

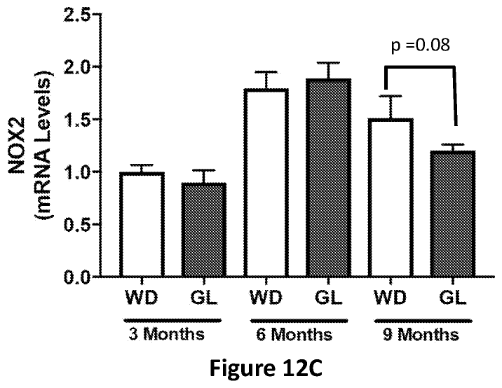

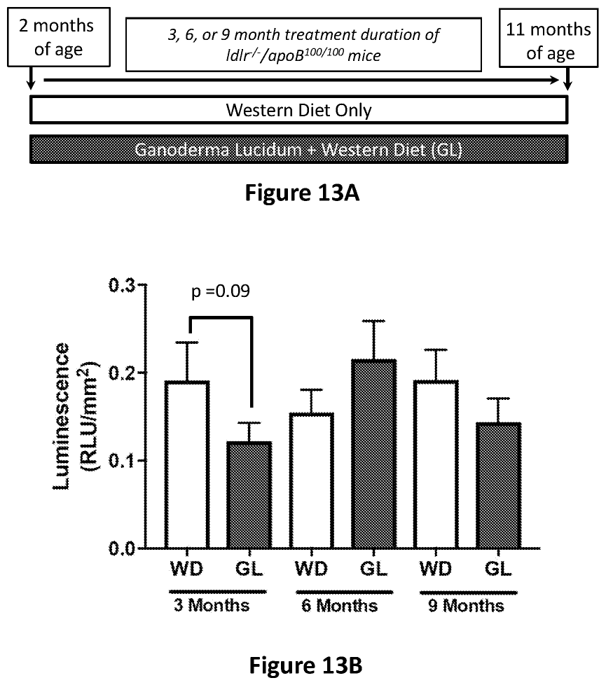

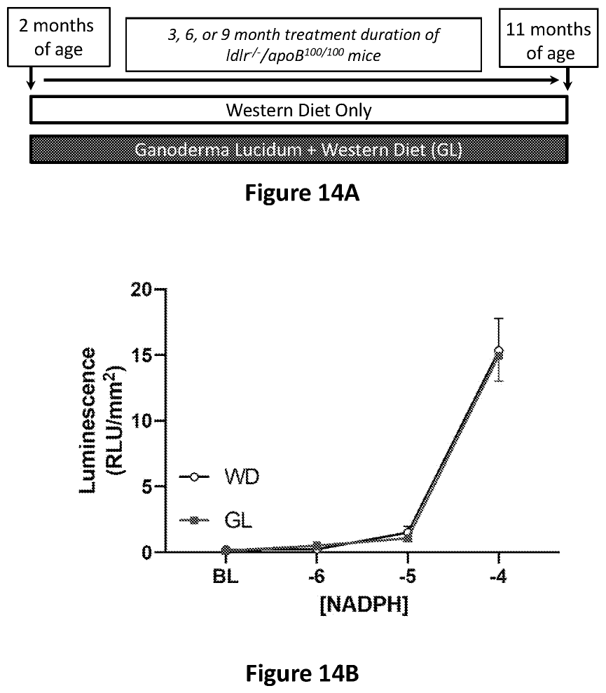

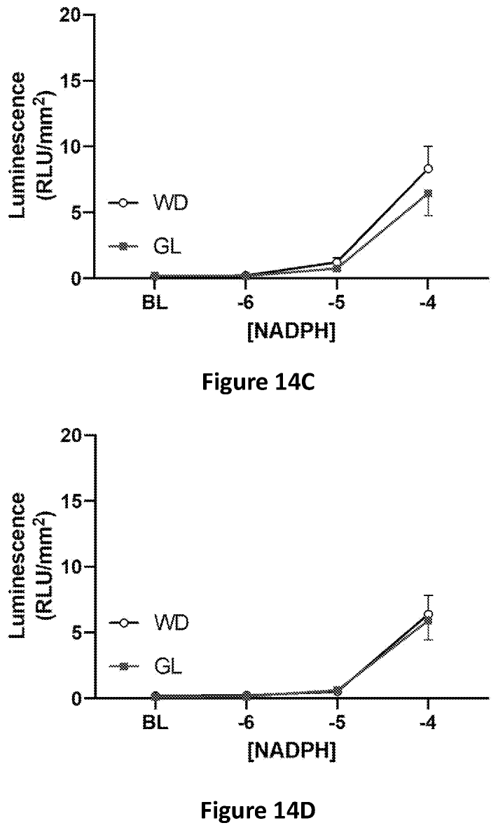

Expression of endothelial nitric oxide synthase (eNOS) is a major promoter of nitric oxide bioavailability and endothelium-independent relaxation, which is countered in disease by increases in NADPH oxidase-derived free radicals (the NOX2 isoform in particular). Measurement of expression of eNOS revealed that long-term treatment with GL did not increase eNOS expression significantly in early, mid, or late stage atherosclerosis (FIG. 12B). Measurement of Nox2 expression did reveal, however, that expression of this deleterious gene was modestly reduced following 9 months of GL treatment (compared to 9 months of WD, FIG. 12C). Assessment of reactive oxygen species levels using lucigenin-enhanced chemiluminescence also suggested that GL may reduce both basal reactive oxygen species levels in early disease (FIG. 13B following 3 months of treatment) and reduce NADPH oxidase activity in later stages of disease (FIG. 14C following 6 months of treatment). This suggests that long-term treatment with GL may favor reduced reactive oxygen species production and improved endothelium-dependent relaxation in atherosclerosis, although the magnitude of such changes may make it a minor mechanism contributing to the observed dramatic improvements in vascular function (FIG. 6).

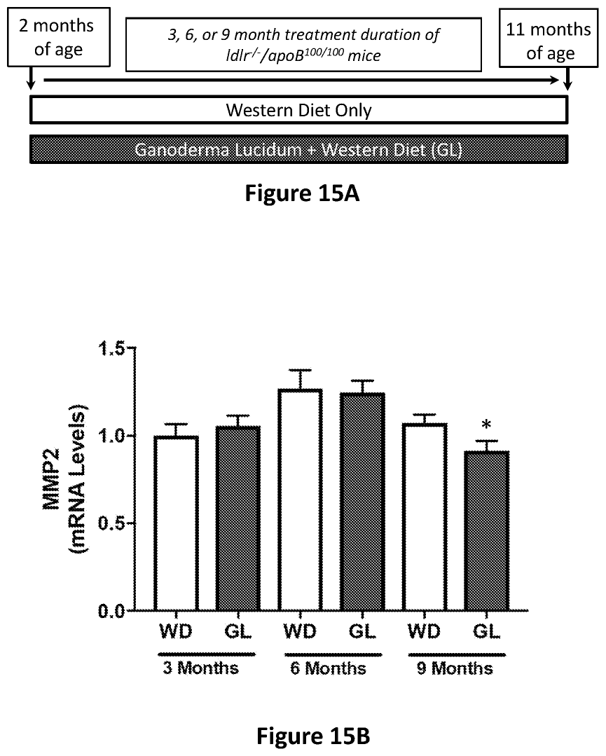

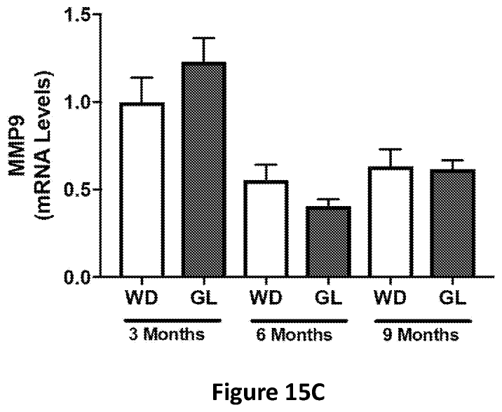

Changes in matrix metalloproteinase isoforms are a major contributor to changes in collagen fiber thickness, tissue stiffness, and plaque susceptibility to rupture due to collagen degradation. Measurement of expression of MMP2 and MMP9 in aorta from GL-treated mice revealed that long-term treatment with GL reduced MMP2 expression significantly in late stages of disease (FIG. 15B following 9 months of treatment, p<0.05). Expression of MMP9 in the same tissues was not consistently or significantly changed by long-term administration of GL. This suggests that long-term treatment with GL may be a viable strategy to reduce excess matrix remodeling in cardiovascular tissues and reduce risk of cardiovascular events.

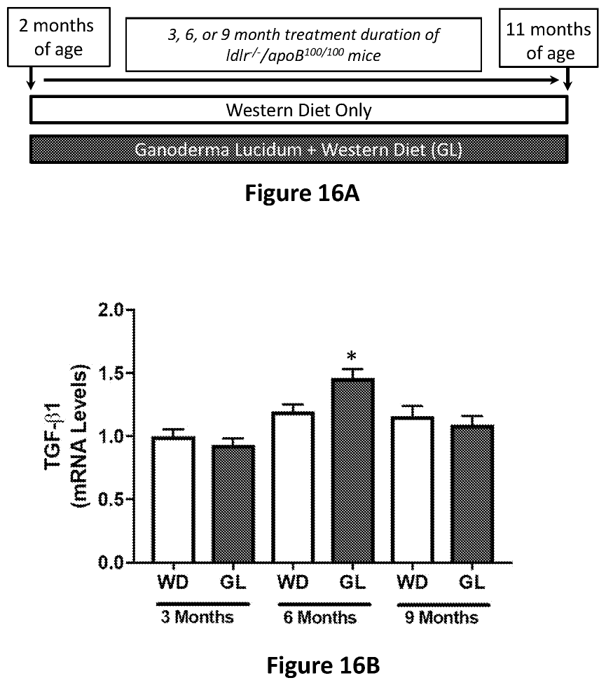

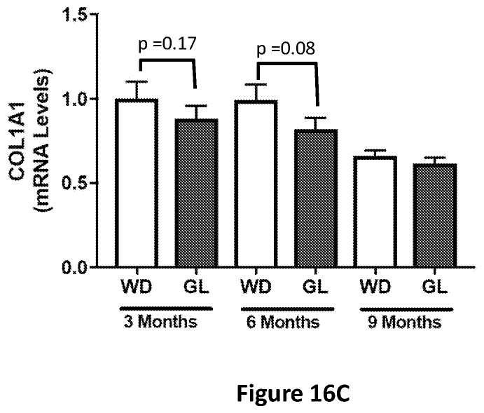

Transforming growth factor beta-1 is a master regulator of tissue fibrosis and matrix remodeling, and can also regulate cell proliferation and inflammation in a context-dependent manner. Measurement of TGFbeta-1 in aorta revealed that long-term treatment with GL increases TGFbeta1 expression in intermediate/moderate stages of atherosclerosis (FIG. 16B following 6 months of treatment, p<0.05), which may serve to suppress inflammation. Expression of the downstream TGFbeta-1 target gene collagen 1A1 (COL1A1) was reduced in early stages of disease (FIG. 16 C at both 3 and 6 month time points) in GL-treated mice, which was consistent with histological data showing reductions in collagen fiber thickness in intimal plaques with GL treatment (FIG. 9). Collectively, these data suggest that treatment with GL may be a viable strategy to augment TGFbeta-1 signaling and harness its protective effects while concomitantly reducing pathological collagen dynamics/turnover during progression of atherosclerosis.

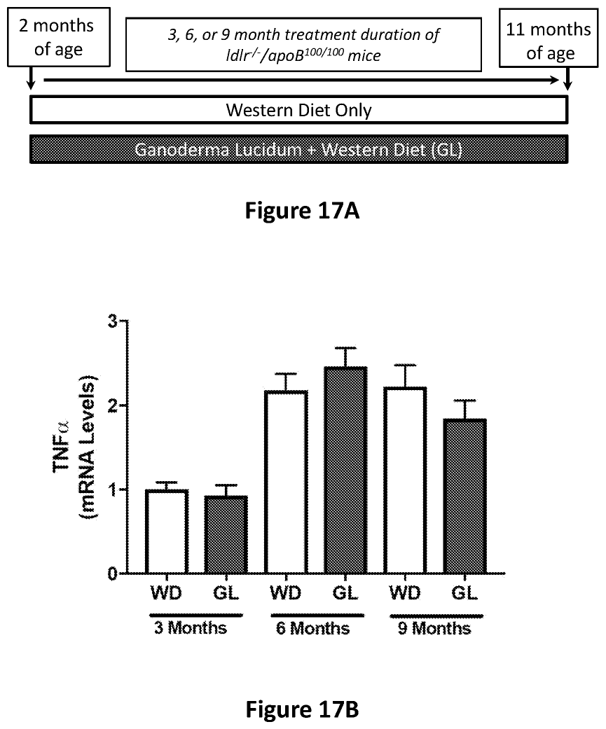

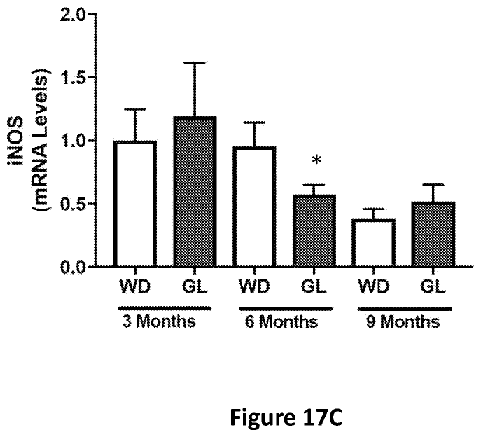

Inflammation is a major driver of plaque expansion and destabilization in atherosclerosis, and is also strongly implicated in accelerated cardiovascular stiffening with increasing age. Measurement of TNFα in aortic tissue from hypercholesterolemic mice revealed that long-term treatment with GL does not significantly reduce expression of this upstream, key factor driving inflammation (FIG. 17B). Measurement of iNOS, however, revealed that GL reduced expression of this pro-inflammatory gene in moderate/intermediate atherosclerosis (FIG. 17C, p<0.05). This suggests that long-term treatment of GL may prove to be efficacious in reducing inflammatory signaling at specific stages of atherosclerotic disease.

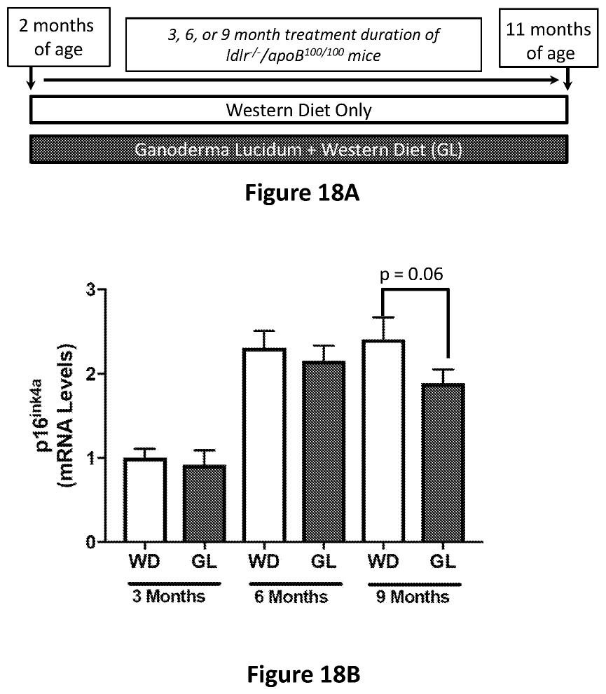

Cellular senescence is thought to be a major contributor to accelerated organismal aging and emergence multiple pathological age-associated phenotypes (cardiovascular diseases and other conditions). Measurement of p161^{ink4a}, a key marker of senescence cell burden-showed that senescence increases with WD over time, and that treatment with GL can reduce senescent cell burden in advanced atherosclerosis (FIG. 18B, 9 month treatment). This suggests that GL may function as a senolytic drug and could be used to prevent multiple age-associated cardiovascular diseases, as well as a multitude of other age-associated, senescence-associated, chronic morbidities and/or other disease conditions.

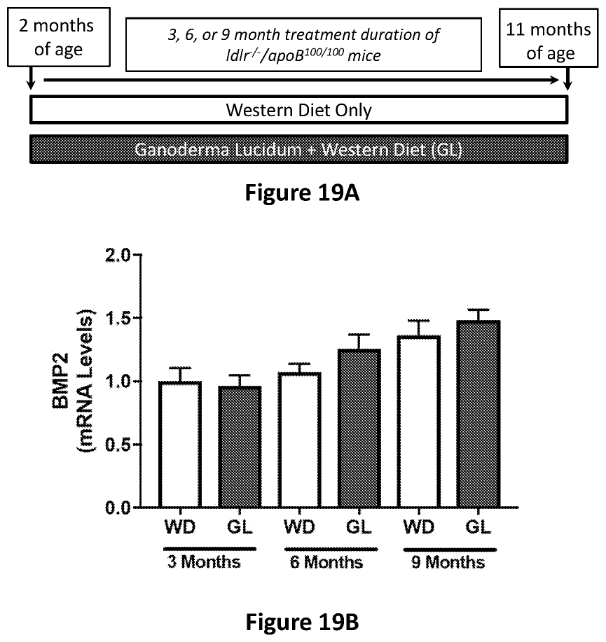

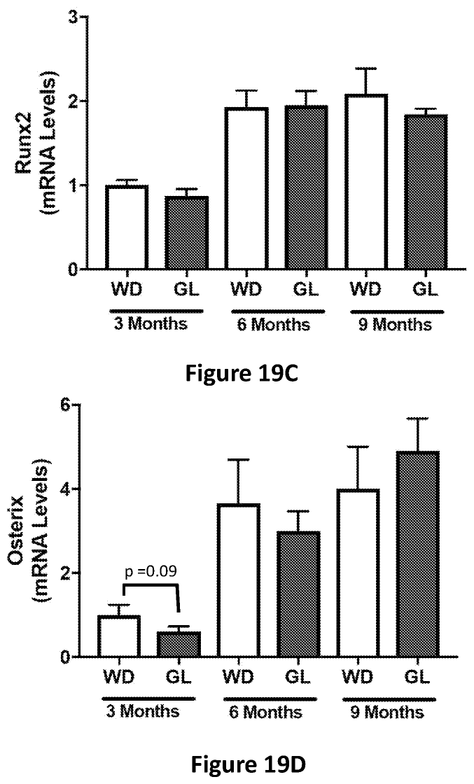

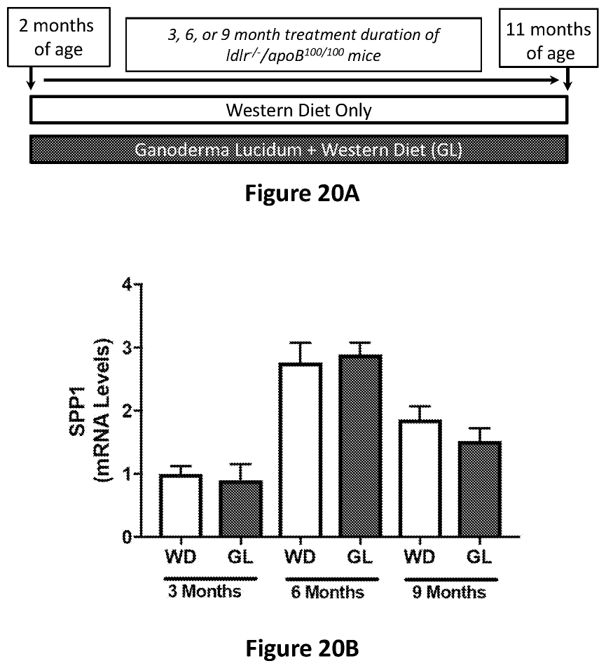

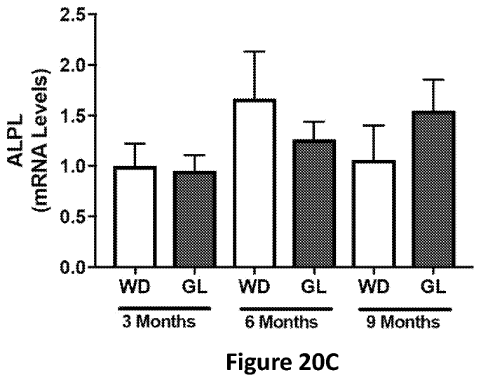

Numerous lines of evidence suggest that cardiovascular calcification can be induced by osteogenic and non-osteogenic mechanisms at various sites (aorta, aortic valve, microvessels, etc.), and preferential targeting of these mechanisms is likely to drive development of novel strategies to slow progression of calcification within complex plaques. Measurement of BMP2 (a major driver of calcification in cardiovascular tissues, FIG. 19B), Runx2 (a master regulator of osteogenesis, FIG. 19C), and Osterix (a transcription factor often induced by BMP2 signaling) revealed that GL does not reduce osteogenic signaling in moderate to severe vascular disease. These data also reveal that, while GL does not reduce BMP2 (FIG. 19B) or Runx2 (FIG. 19C) in early disease (i.e., following 3 months of treatment), GL treatment does tend to reduce osterix expression in early disease (FIG. 19D). Measurement of additional genes related to osteogenesis-driven calcification-such as osteopontin (SPP1 in FIG. 20B) and alkaline phosphatase (ALPL in FIG. 20C) in vascular tissues provided further support for a lack of influence of GL on osteogenic signaling in advanced atherosclerotic disease. Collectively, these data suggest that GL may selectively modulate some osteogenic signaling factors in very early disease, but is not likely to be a primary mechanism contributing to GL-driven reductions in calcification in advanced disease (i.e., GL may reduce calcification by non-osteogenic mechanisms). Importantly, this also suggests that GL is likely to reduce calcification in cardiovascular tissue (e.g., FIG. 10C) but not negatively influence bone ossification/bone mineral density by interfering with conserved mechanisms of ectopic and orthotopic ossification.

Assessments of the Left Ventricle Following Treatment with WD+GL

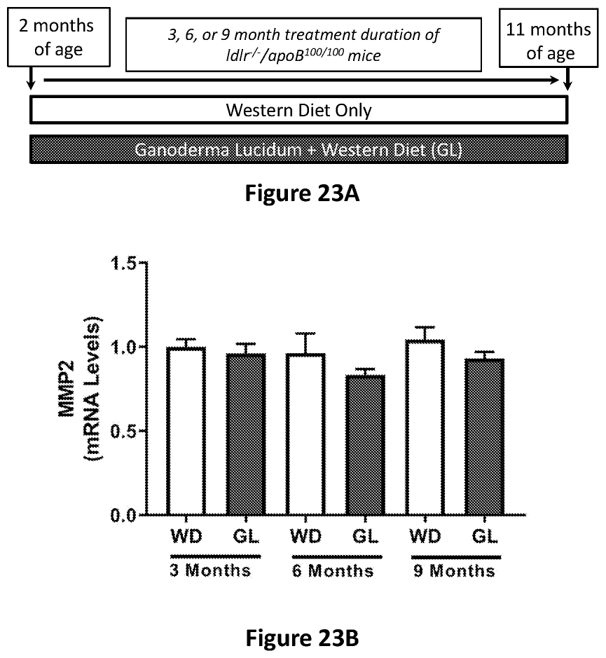

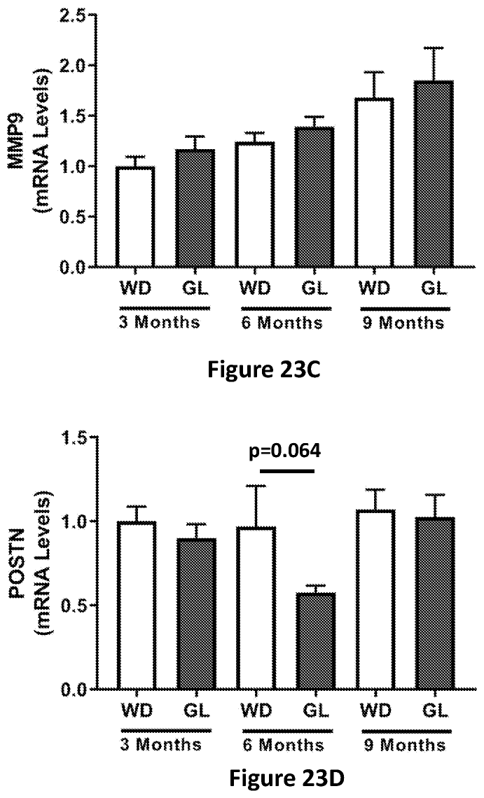

In addition to the assessment overall left ventricular function, fibrosis of the left ventricular was also analyzed. As mentioned above, changes in matrix metalloproteinase isoforms are a major contributor to changes in collagen fiber thickness, tissue stiffness, and plaque susceptibility to rupture due to collagen degradation. Measurement of expression of MMP2 and MMP9 in left ventricles from GL-treated mice revealed that long-term treatment with GL reduced MMP2 expression in late stages of disease (FIG. 23B show decreases in expression at 6 months and 9 months of treatment). Expression of MMP9 in the left ventricle was slightly increased compared to mice receiving only a Western Diet (FIG. 23C). In addition, periostin (POSN), which is a secreted extracellular matrix protein that functions in tissue development and regeneration, including wound healing, and ventricular remodeling following myocardial infarction, was altered at an intermediate stage of disease (FIG. 23D). This suggests that long-term treatment with GL does not change fibrosis of the left ventricle and therefore may be a viable strategy to reduce excess matrix remodeling in cardiovascular tissues and reduce risk of cardiovascular events.

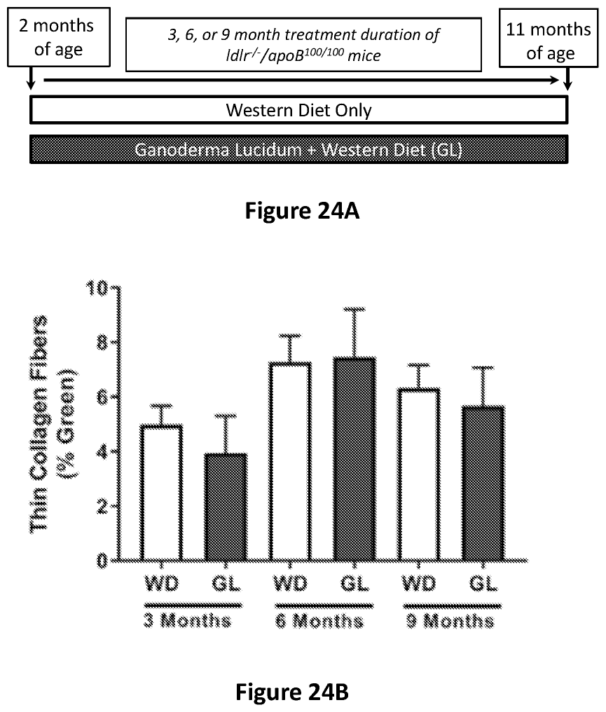

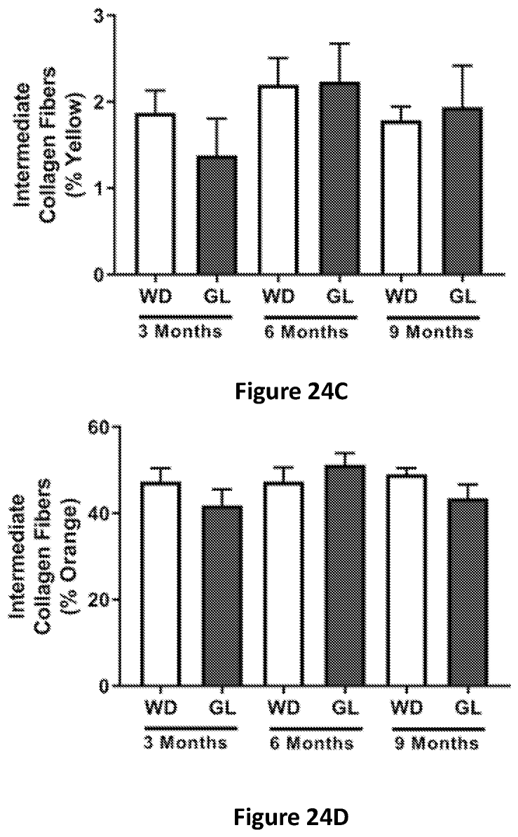

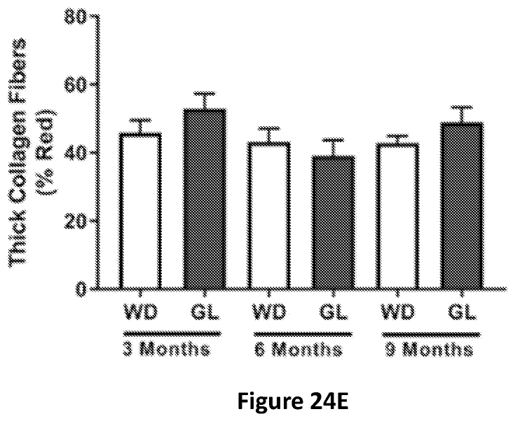

Thickness of collagen fibers can be a major contributor to both plaque stiffness (which can augment vascular calcification) and can also be a significant determinant of risk for plaque rupture and cardiovascular events. As seen in FIG. 24A, and as performed for the experiments in FIG. 9, measuring collagen fiber thickness using picrosirius red staining and circularly polarized light imaging (which allows for assessment of relative collagen fiber thicknesses) in FIG. 24B-E revealed that long-term treatment with GL does not significantly alter the thickness. The proportion of thin collagen fibers in the left ventricle did not change throughout disease progression. Therefore, taken together with improvements in other measurements of cardiovascular stiffness (e.g., left ventricular diastolic stiffness in FIG. 4) suggests a broader impact of GL on cardiovascular stiffness and plaque stability in multiple tissues.

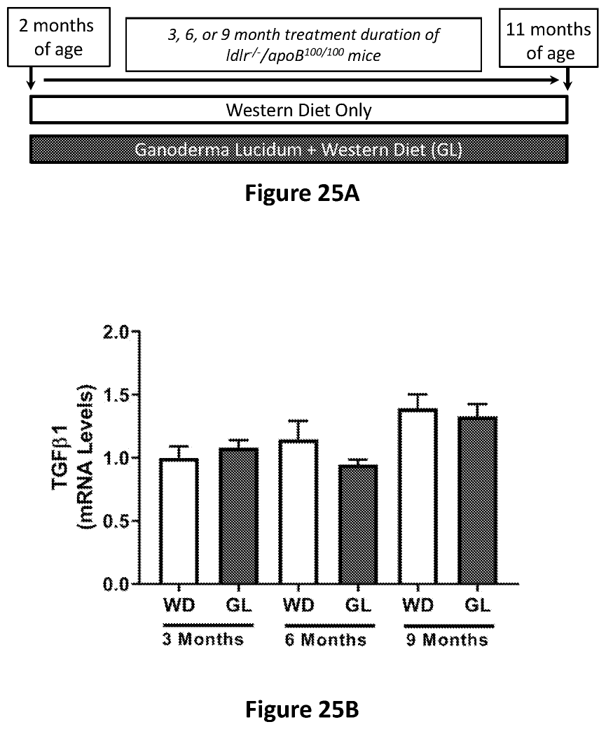

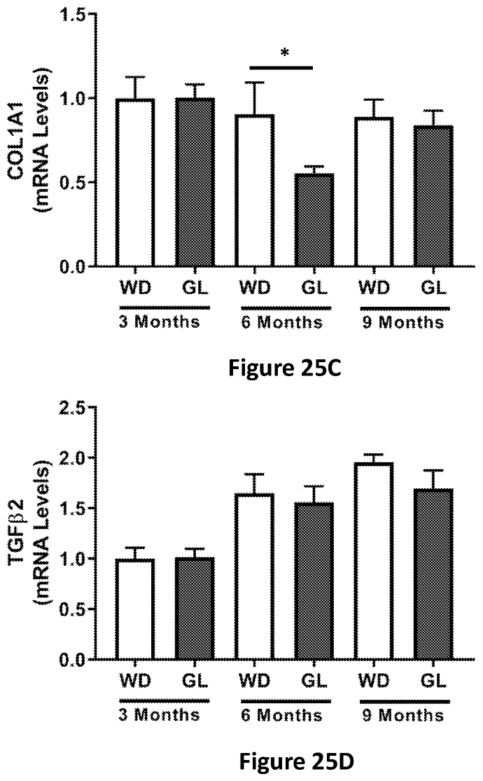

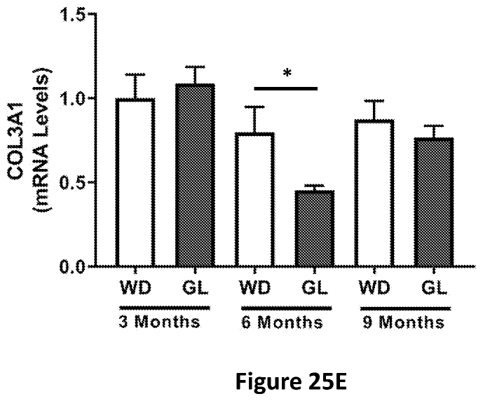

As mentioned above, transforming growth factor beta-1 is a master regulator of tissue fibrosis and matrix remodeling. Measurement of TGFbeta-1 in left ventricles revealed that long-term treatment with GL does not significantly alter expression of TGFbeta1 or TGFbeta2 expression at any stage of atherosclerosis (FIGS. 25B and 25D), which may serve to suppress inflammation. Interestingly, expression of downstream target genes of TGFbeta signaling including collagen 1A1 (COL1A1) and collagen 3A1 (COL3A1) were both reduced in intermediate stages of the disease (FIGS. 25C and 25E at 6 month time points) in GL-treated mice. Collectively, these data suggest that treatment with GL may be a viable strategy to augment TGFbeta-1 and/or TGFbeta-2 signaling and harness the protective effects of TGFbeta signaling.

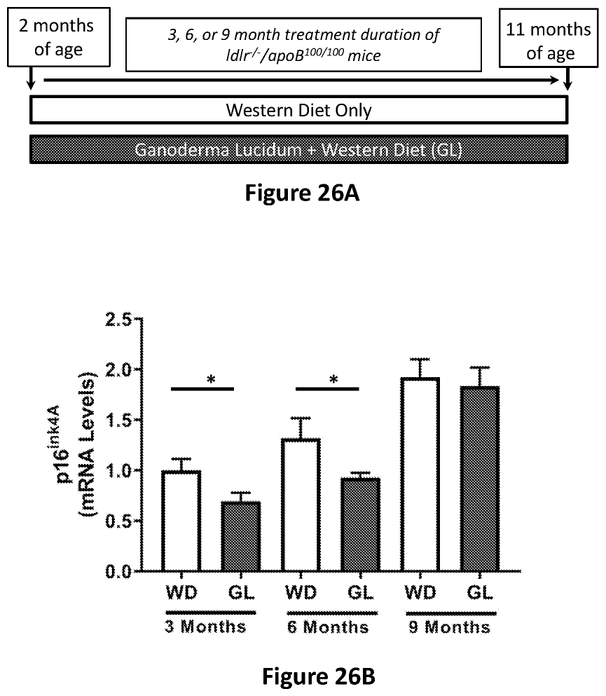

Finally, in order to determine cellular senescence in left ventricles in mice treated with WD or WD+GL, expression of p16ink4a, a key marker of cellular senescence, was measured. Here, p16ink4a expression showed that senescence increases with WD over time, and that treatment with GL can reduce senescent cell burden in the left ventricle during atherosclerosis (FIG. 26B, 3 month treatment and 6 month treatment). These results demonstrate that GL can function as a senolytic drug and can be used to prevent multiple age-associated cardiovascular diseases.

### Example 2—Administration of Ganoderma lucidum Extract to a Human Identified as have Cardiovascular Disease

A human patient is identified as having cardiovascular disease based on the results of an electrocardiogram (ECG) and is determined to be in need of treatment with a composition containing a GL extract. A 200 mg dose of pharmaceutical composition containing a GL extract is orally administered to the patient. Over the course of the treatment, the patient's symptoms are monitored using ECG. Results of the ECG show a reduction in the symptoms. After each ECG check-up, dose and frequency of administration are assessed, but no changes are made. Given the successful reduction of symptoms, treatment is continued with the aim of re-assessing dosage after elimination of symptoms.

### Example 3—Administration of Ganoderma lucidum Extract to a Human to Slow Age-Associated Cardiovascular Dysfunction

A human patient is identified as being in need of treatment with a composition containing a GL extract to slow the development of age-associated, acquired, or congenital cardiovascular dysfunction within the patient. A daily dosage of 200 mg of pharmaceutical composition containing a GL extract is administered orally to the identified human patient. The patient is maintained on this treatment several months to years (in some cases, for their remaining life) to slow the development of age-associated cardiovascular dysfunction.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-7. (canceled)

8. A method for treating a mammal having cardiovascular disease, wherein said method comprises administering a composition comprising a Ganoderma lucidum extract to a mammal identified as having or as being at risk of developing a cardiovascular disease that comprises one or more symptoms that are responsive to treatment with said composition.

9. The method of claim 8, wherein said mammal is a human.

10. The method of claim 8, wherein said cardiovascular disease is age-related cardiovascular dysfunction.

11. The method of claim 8, wherein said cardiovascular disease is acquired cardiovascular dysfunction.

12. The method of claim 8, wherein said cardiovascular disease is congenital cardiovascular dysfunction.

13-14. (canceled)

15. A method for slowing development of age-related cardiovascular dysfunction, wherein said method comprises administering a composition comprising a Ganoderma lucidum extract to a mammal identified as being in need of a treatment to slow development of said age-related cardiovascular dysfunction.

16. The method of claim 15, wherein said mammal is a human.

17. The method of claim 15, wherein said mammal that was identified has one or more symptoms of age-related cardiovascular dysfunction responsive to treatment with said composition.

18-19. (canceled)

20. A method for slowing development of acquired cardiovascular dysfunction, wherein said method comprises administering a composition comprising a Ganoderma lucidum extract to a mammal identified as being in need of a treatment to slow development of said acquired cardiovascular dysfunction.

21. The method of claim 20, wherein said mammal is a human.

22. The method of claim 20, wherein said mammal that was identified has one or more symptoms of acquired cardiovascular dysfunction responsive to treatment with said composition.

23-24. (canceled)

25. A method for slowing development of congenital cardiovascular dysfunction, wherein said method comprises administering a composition comprising a Ganoderma lucidum extract to a mammal identified as being in need of a treatment to slow development of said congenital cardiovascular dysfunction.

26. The method of claim 25, wherein said mammal is a human.

27. The method of claim 25, wherein said mammal that was identified has one or more symptoms of congenital cardiovascular dysfunction responsive to treatment with said composition.
